(19) United States
(12) Patent Application Publication
(10) Pub. No.: US 2023/0285676 A1
(43) Pub. Date: Sep. 14, 2023

(54) PRE-FILLED MULTI-FLUID MEDICAL DELIVERY ASSEMBLIES

(71) Applicant: Koska Family Limited, East Sussex (GB)

(72) Inventors: Marc Andrew Koska, Eastbourne (GB); Max Hannon, Flemingate (GB); Harris Ryder, Redruth (GB)

(21) Appl. No.: 18/141,559

(22) Filed: May 1, 2023

Related U.S. Application Data
(63) Continuation of application No. PCT/IB2021/059993, filed on Oct. 28, 2021.
(60) Provisional application No. 63/107,155, filed on Oct. 29, 2020.

(51) Int. Cl. A61M 5/24; A61M 5/28

(52) U.S. Cl. CPC A61M 5/2448; A61M 5/284

(57) ABSTRACT

A pre-filled medical delivery assembly can have a blow-fill-seal (BFS) module, a manifold, and a casing. The BFS module can have a pair of reservoirs, and a pair of sealed ports. Each reservoir can have a respective liquid agent therein. Each port can be in fluid communication with a respective one of the reservoirs. Part of the BFS module can be inserted into the manifold, and the casing can protect part of the BFS module exposed from the manifold. An orientation of the casing can be reversed, and the casing can be used to push the BFS module into the manifold to breach the seals and/or to compress the reservoirs to dispense the liquid agents. The disclosed assemblies can combine the liquid agents from the BFS module and deliver the combination as a single dose of a therapeutic agent (e.g., vaccine, drug, medicament, etc.) to a patient.

### CLAIM OF PRIORITY

This application is a Continuation application of PCT Application No. PCT/IB2021/059993, filed on Oct. 28, 2021 in the name of Koska et al. and titled PRE-FILLED MULTI-FLUID MEDICAL DELIVERY ASSEMBLIES, which PCT Application claims benefit of and priority under 35 U.S.C. § 119(e) to, and is a Non-provisional of U.S. Provisional Patent Application No. 63/107,155 filed on Oct. 29, 2020 and titled PRE-FILLED DUAL-LIQUID MEDICAL DELIVERY ASSEMBLIES. Each of these applications is hereby incorporated by reference herein in its entirety and for all purposes.

### STATEMENT REGARDING FEDERALLY SPONSORED RESEARCH

This invention was made with government support under Award No. 75A50120C00007, awarded by the Department of Health and Human Services (HHS). The government has certain rights in the invention.

### BACKGROUND

Every year, millions of people become infected and die from a variety of diseases, some of which are vaccine-preventable. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs include administration of vaccines via a reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting in the death of more than one million people each year.

Previous attempts at providing single-use or disposable injection devices to remedy such problems in the industry have achieved measurable success but have failed to adequately remedy the existing problems. Pre-filled, single-use injection devices manufactured via injection molding or Form-Fill-Seal (FFS) processes, such as the Uniject™ device available from the Becton, Dickinson and Company of Franklin Lakes, N.J., for example, while offering precise manufacturing tolerances in the range of two thousandths of an inch (0.002-in; 50.8 μm) to four thousandths of an inch (0.004-in; 101.6 μm)—for hole diameters in molded parts, require separate sterilization processes (e.g., gamma radiation) that are not compatible with certain fluids, provide production rates limited to approximately nine thousand (9,000) non-sterile units per hour, and can be provided to an end-user for approximately one dollar and forty cents ($1.40) per dose/unit. Such devices also are not configured to store, mix, and/or deliver a medicament comprising multiple liquids such as a vaccine and an adjuvant.

### SUMMARY

Embodiments of the disclosed subject matter provide systems, assemblies, kits, and methods for medical delivery of liquid agents to a patient. Using a blow-fill-seal (BFS) fabrication technique, a BFS module (such as a vial or other component) is pre-filled with respective fluid (e.g., liquid) agents, which are maintained in separate sealed reservoirs within the BFS module, for example, until ready for use. In some embodiments, part of the BFS module can be inserted into a manifold, which includes piercing elements for breaching seals of the BFS module. Necks of the BFS module can interact with mating surfaces within the manifold to retain sealed ports of the BFS module spaced from the piercing elements, for example, until such time that combination of the liquid agents and dispensing is desired. A protective casing can be provided over part of the BFS module exposed from the manifold. To breach the seals, the protective casing can be removed, and its orientation reversed, such that a narrowed recess thereof can be advanced longitudinally over the part of the BFS module exposed from the manifold. Pushing the narrowed recess onto the BFS module can initially push the BFS module further into the manifold such that the piercing elements breach the seals. Further pushing of the narrowed recess over the part of the BFS module exposed from the manifold can compress the reservoirs to dispense the fluid agents therein into a mixing chamber of the manifold and subsequently out of the manifold for administration. The fluid agents from the separate reservoirs can thus be combined prior to administration to a patient. In some embodiments, the combined fluid agents are administered via a needle or cannula that is part of or coupled to the manifold, or via a nozzle that is part of or coupled to the manifold. For example, embodiments of the disclosed subject matter can deliver the combination of the liquid agents from the BFS module as a single dose of a therapeutic agent (e.g., vaccine, drug, medicament, etc.).

In one or more embodiments, a pre-filled medical delivery assembly can comprise a BFS module. The BFS module can have first and second longitudinal ends, and can comprise first and second reservoirs and first and second sealed ports. The first reservoir can have a first liquid agent therein, and the second reservoir can have a second liquid agent therein. The first and second sealed ports can be proximal to the first longitudinal end of the BFS module. The first sealed port can be in fluid communication with the first reservoir, and the second sealed port can be in fluid communication with the second reservoir.

In some embodiments, the pre-filled medical delivery assembly can further comprise a manifold. The manifold can have third and fourth longitudinal ends. The manifold can further have an internal volume extending from the third longitudinal end to the fourth longitudinal end. A portion of the internal volume proximal to the third longitudinal end can be constructed to act as a mixing chamber for the first and second liquid agents. The first longitudinal end of the BFS module can be received (or constructed to be received) through the fourth longitudinal end of the manifold with at least part of the first and second reservoirs exposed from the manifold. The manifold can further comprise first and second longitudinally-extending piercing elements disposed within the internal volume and respectively aligned with the first and second sealed ports.

In some embodiments, the pre-filled medical delivery assembly can further comprise a protective casing. The protective casing can be disposed over (or constructed to be disposed over) the exposed part of the first and second reservoirs of the BFS module. The protective casing can be releasably coupled to the manifold. In some embodiments, the protective casing can be reversible and can be used to push the BFS module further into the manifold to breach the sealed ports and/or to compress the reservoirs of the BFS module to dispense the liquid agents from the BFS module and/or the manifold. Alternatively, in some embodiments, the protective casing is removed and replaced with an actuation casing that is used to push the BFS module and/or compress the reservoirs. In some embodiments, the protective casing or the actuation casing may be reusable with other BFS modules and/or manifolds. In some embodiments, the BFS module may be disposable, for example, after administration of the combined liquid agents to the patient. In some embodiments, both the BFS module and manifold may be disposable.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Where applicable, some elements may be simplified or otherwise not illustrated in order to assist in the illustration and description of underlying features. For example, in some figures, some components have been illustrated using a partial or cutaway view in order to illustrate internal interaction of components. Throughout the figures, like reference numerals denote like elements. An understanding of embodiments described herein and many of the attendant advantages thereof may be readily obtained by reference to the following detailed description when considered with the accompanying drawings, wherein:

FIGS. 1A-1B are simplified schematic diagrams of a pre-filled medical delivery assembly prior to and after breach of sealed ports of a blow-fill-seal (BFS) module, respectively, according to one or more embodiments of the disclosed subject matter;

FIG. 1C is a simplified schematic diagram of the pre-filled medical delivery assembly of FIGS. 1A-1B after partial reservoir compression by a casing to dispense liquid agents from the BFS module into a mixing chamber, according to one or more embodiments of the disclosed subject matter;

FIG. 1D is a simplified schematic diagram of the pre-filled medical delivery assembly of FIG. 1C after further reservoir compression by a casing to administer the combined liquid agents to a patient, according to one or more embodiments of the disclosed subject matter;

FIGS. 2A-2C are top, front, and side views, respectively, of an exemplary BFS module, according to one or more embodiments of the disclosed subject matter;

FIGS. 3A-3D are perspective, top, sectional perspective, and side cross-sectional views, respectively, of an exemplary manifold, according to one or more embodiments of the disclosed subject matter;

FIGS. 4A-4B are perspective views of first and second orientations, respectively, of an exemplary casing, according to one or more embodiments of the disclosed subject matter;

FIGS. 4C-4F are top, front, bottom, and side views, respectively, of the casing of FIGS. 4A-4B, according to one or more embodiments of the disclosed subject matter;

FIGS. 5A-5B are assembled and longitudinally-exploded views, respectively, of an exemplary pre-filled medical delivery assembly, according to one or more embodiments of the disclosed subject matter;

FIGS. 6A-6B are perspective and side cross-sectional views, respectively, of the exemplary pre-filled medical delivery assembly of FIGS. 5A-5B in an initial assembled state, according to one or more embodiments of the disclosed subject matter;

FIG. 7A-7B are perspective and side cross-sectional views, respectively, of the exemplary pre-filled medical delivery assembly of FIGS. 6A-6B with the casing removed for reorientation, according to one or more embodiments of the disclosed subject matter;

FIG. 8A-8B are perspective and side cross-sectional views, respectively, of the exemplary pre-filled medical delivery assembly of FIGS. 7A-7B with the reoriented casing being positioned to actuate the BFS module, according to one or more embodiments of the disclosed subject matter;

FIGS. 9A-9C are perspective, detailed cross-sectional, and side views, respectively, of the exemplary pre-filled medical delivery assembly of FIGS. 8A-8B after initial displacement of the casing to cause breaching of the sealed ports of the BFS module, according to one or more embodiments of the disclosed subject matter;

FIG. 10A-10B are perspective and cross-sectional views, respectively, of the exemplary pre-filled medical delivery assembly of FIGS. 9A-9B after removal of needle cap, according to one or more embodiments of the disclosed subject matter;

FIG. 11A-11B are perspective and cross-sectional views, respectively, of the exemplary pre-filled medical delivery assembly of FIGS. 10A-10B in preparation for injection, according to one or more embodiments of the disclosed subject matter;

FIGS. 12A-12B are perspective and cross-sectional views, respectively, of the exemplary pre-filled medical delivery assembly of FIGS. 11A-11B after further displacement of the casing to cause dispensing of the liquid agents into the mixing chamber of the manifold, according to one or more embodiments of the disclosed subject matter;

FIGS. 13A-13B are perspective and cross-sectional views, respectively, of the exemplary pre-filled medical delivery assembly of FIGS. 12A-12B after further displacement of the casing to cause the combined liquid agents to be dispensed from the mixing chamber into a patient, according to one or more embodiments of the disclosed subject matter;

FIGS. 14A-14B are perspective and cross-sectional views, respectively, of the exemplary pre-filled medical delivery assembly of FIGS. 13A-13B after administration, according to one or more embodiments of the disclosed subject matter; and

FIG. 15 is a process flow diagram of an exemplary method for assembly and use of a pre-filled medical delivery assembly for administration of combined liquid agents, according to one or more embodiments of the disclosed subject matter.

### DETAILED DESCRIPTION

### I. Introduction

Described herein are systems, assemblies, kits, and methods for medical delivery of multiple fluid (e.g., liquid) agents (e.g., at least two) to a patient (e.g., human or animal) from a pre-filled (and, in some embodiments, field-assembled or assembled at the point-of-use) module (also referred to herein as a component, bottle, or vial). In some embodiments, at least two liquid agents are sealed within the module and retained separate from each other until a time when it is desirable to mix the liquid agents (e.g., at the time of or prior to administration to the patient). The liquid agents can be any type of agent to be injected into or otherwise delivered to a patient and capable of producing a therapeutic effect, either alone or in combination with an active ingredient. Accordingly, the liquid agents can include, but are not limited to, separate vaccines, drugs, medicaments, diluents, active ingredients, etc. that are desirable to combine for common administration to the patient. For example, in some embodiments, the combination of the liquid agents can form a multi-fluid agent, solution, mixture, suspension, etc. Alternatively or additionally, the liquid agents can be separate components that, when combined, form a vaccine, drug, medicament, etc. For example, in some embodiments, the combination of the liquid agents can comprise a single dose of a therapeutic agent (e.g., vaccine, drug, medicament, etc.). In some embodiments, one or more of the liquid agents in the vial can be tracked, monitored, checked for compatibility, etc., such as by utilization of electronic data storage devices (not shown) coupled to the various modules or components of the delivery system.

In some embodiments, the component may comprise at least one blow-fill-seal (BFS) module that has multiple chambers prefilled with respective liquid agents using a BFS manufacturing technique. At least some of the chambers filled with liquid agents can be sealed from other of the chambers, thereby maintaining the liquid agents separate until combination thereof is desired (e.g., a time for administration to the patient). The BFS module may be constructed, filled, and sealed, according to some embodiments, in a sterile manufacturing environment. BFS modules may, for example, offer a less expensive alternative to typical vials or bottles created via other manufacturing techniques. In some embodiments, BFS modules (e.g., due to the nature of the BFS manufacturing process) may not require separate sterilization (e.g., and may accordingly be compatible with a wider array of liquid agents), may provide enhanced production rates of sterile/aseptic units per hour, and/or may be provided to an end-user for significantly lower per dose/unit costs. In some embodiments, these advantages may come with an attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989), which is incorporated herein by reference.

In some embodiments, a manifold (also referred to herein as a mixing assembly, mixing connector, or multi-port coupling) can be coupled to the BFS module to breach seals of the BFS module and to provide fluid communication between the respective chambers to allow mixing of liquid agents external to the BFS module prior to administration. For example, in some embodiments, the combination (e.g., mixing) can occur within the manifold. The pre-filled multi-fluid medical delivery assemblies of some embodiments may include a specially-designed multi-chamber BFS module coupled into a specialized collar of the manifold, which can facilitate coupling of an administration assembly (e.g., a needle) to the BFS module.

In some embodiments, a removable casing (also referred to herein as protective casing, housing, covering, or “hard pack”) may be operable to (i) protect the BFS module (e.g., with the casing in a first orientation) and (ii) facilitate or guide activation of the medical delivery assembly (e.g., with the casing in a second orientation). According to some embodiments, a pre-filled multi-fluid medical delivery assembly may be selectively actuated by displacing the BFS module within the manifold (e.g., by applying longitudinal force via the removable casing) that causes piercing of the BFS module chambers. In some embodiments, the removable casing can be further displaced over the BFS module to cause the chambers to be compressed and emptied.

In some embodiments, the combined liquid agents can be administered to a patient via an administration assembly coupled to the manifold. In some embodiments, the administration assembly comprises a needle or cannula constructed for subcutaneous, intramuscular, intradermal, or intravenous injection of the combined liquid agents into the patient. Alternatively, in some embodiments, the administration assembly comprises a nozzle. For example, the nozzle can be a spray nozzle that facilitates dispersion of the combined liquid agents into a spray, which configuration may be useful in the administration of the combined liquid agents into a body cavity or orifice (e.g., nasal passage, ear canal, etc.). In another example, the nozzle can be a droplet nozzle that facilitates formation of droplets of the combined liquid agents, which configuration may be useful in the administration of the combined liquid agents to the eyes, for topical application, etc. Alternatively, in some embodiments, the combined liquid agents can be administered without coupling of a separate administration assembly, for example, where a needle, cannula, or nozzle is an integrated part of the mixing assembly (e.g., integrally formed with the manifold).

In some embodiments, the administration assembly, the manifold, and/or the removable casing may be configured to be coupled and/or assembled to the BFS module on-site and/or in the field. Alternatively or additionally, in some embodiments, the administration assembly, the manifold, and/or the removable casing may be coupled and/or assembled to the BFS module in a manufacturing facility and provided to users as a single, pre-assembled medical device (e.g., with the BFS module contained between the manifold and the removable casing, but without breach of the sealed ports of the BFS module). The pre-filled multi-liquid medical delivery device may, for example, be capable of delivering combined liquid agents in a controlled manner and without requiring specialized skill in assembling and/or administering delivery of such agent.

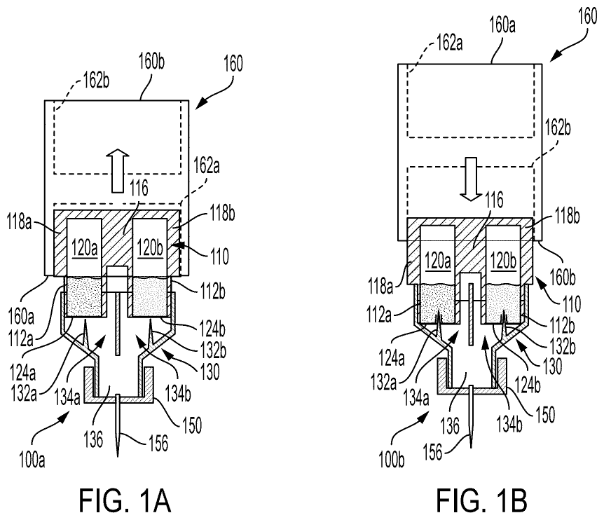

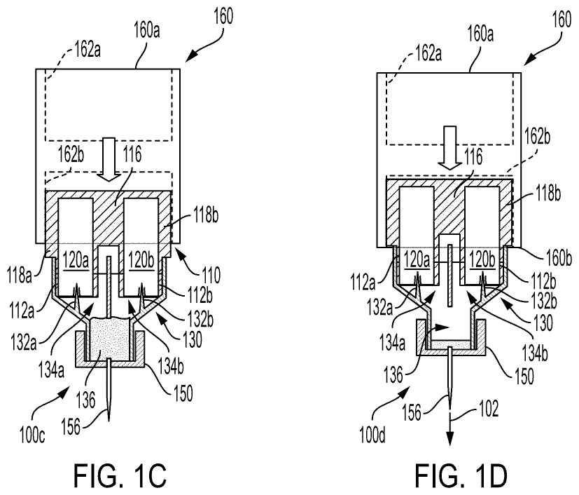

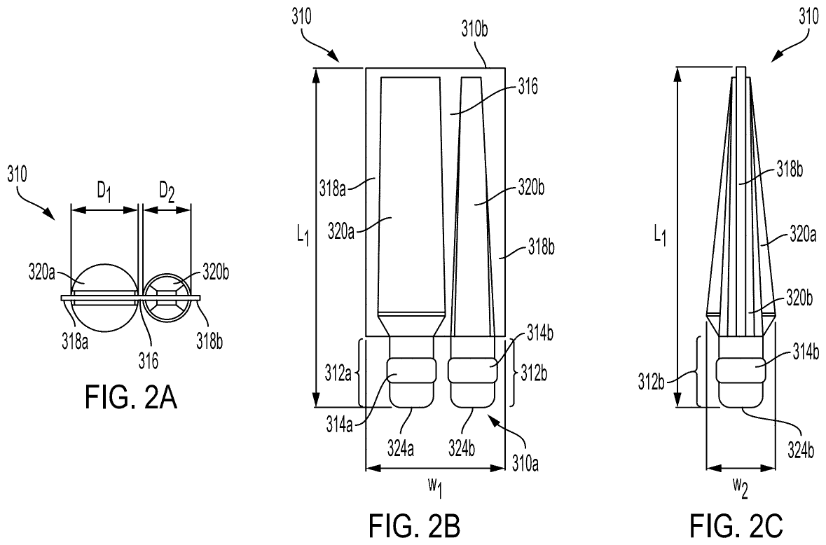

Referring to FIG. 1A, an exemplary pre-filled medical delivery assembly in a first configuration 100 is shown. In some embodiments, the first configuration 100 can be a state of assembly as delivered from a manufacturing facility for use in the field with minimal assembly, or a state of assembly of individual components by a user in preparation for administration. In some embodiments, the medical delivery assembly comprises a BFS module 110. The BFS module 110 can comprise and/or define at least two reservoirs 120a, 120b (also referred to herein as chambers). The reservoirs 120a, 120b can be filled (fully or partially) with a fluid or other agent to be delivered, for example, to a patient. In some embodiments, each reservoir 120a, 120b can be filled with a different fluid, such as a different liquid or gas, and such different fluid agents can be combined, introduced, and/or mixed via the pre-filled multi-liquid medical delivery assembly (e.g., via manifold 130 thereof) to define a fluid agent for delivery to the patient. In some embodiments, a fluid volume within the first reservoir 120a is at least two times greater than a fluid volume within the second reservoir 120b. For example, the first reservoir 120a can house or contain a first volume (e.g., two milliliters (2.0-ml)) of a vaccine or first agent (e.g., Pralidoxime), while a second fluid reservoir 120b may house or contain a second volume (e.g., seven tenths milliliters (0.7-ml)) of an adjuvant (and/or carrier fluid, catalyst, diluent, etc.) or second agent (e.g., Atropine). According to some embodiments, any or all of the fluid agents may be injected or otherwise loaded into the BFS module 110 (e.g., into respective reservoirs 120a, 120b thereof) in a sterile environment during manufacture via a BFS process.

The reservoirs 120a, 120b can be joined together via a central web 116 extending laterally between facing surface portions of the reservoirs. In addition, a first side rib 118a can extend laterally outward from the first reservoir 120a, and a second side rib 118b can extend laterally outward from the second reservoir 120b. In some embodiments, the first side rib 118a, the second side rib 118b, and the central web 116 can be part of a body flange of the BFS module 110. For example, as a result of the BFS molding process, the body flange can be formed extending from the edges of the first and second reservoirs 120a, 120b along the parting line. In some embodiments, the body flange can serve as a connection between adjacent modules simultaneously formed during the molding process. In some embodiments, the BFS module 110 can further comprise and/or define first and second neck portions 112a, 112b (e.g., substantially cylindrical) that extend longitudinally from a longitudinal end of the BFS module 110. Each neck portion 112a, 112b can be in fluid communication with a respective fluid reservoir 120a, 120b and/or can comprise a fluid seal 124a, 124b at a terminal end thereof, e.g., to maintain the fluid agents in the respective reservoirs and/or neck portion 112a, 112b. In some embodiments, the fluid seals 124a, 124b can comprise portions of the molded BFS module 110 itself, for example, molded portions that are constructed to be breached (e.g., pierced) to expel the respective fluids. For example, the fluid seals 124a, 124b can be breached by providing a flat or planar piercing surface and/or by being oriented normal to an axis of the BFS module 110 (and/or the pre-filled multi-liquid medical delivery assembly). Alternatively or additionally, one or both of the fluid seals 124a, 124b can comprise foil, wax, paper, plastic, and/or other thin, pierceable objects or layers formed as part of (or coupled to) the BFS module 110.

In some embodiments, the medical delivery assembly comprises a manifold 130. The manifold 130 can comprise and/or define an internal volume that can extend from one longitudinal end of the manifold 130 (e.g., an open upper end, into which a portion of the BFS module 110 can be inserted) to an opposite longitudinal end of the manifold 130 (e.g., a lower end comprising and/or defining an outlet port 136). In some embodiments, the neck portions 112a, 112b can be inserted into and/or releasably coupled within the internal volume of the manifold 130, with the fluid seals 124a, 124b of the neck portions 112a, 112b facing respective dispensing chambers 134a, 134b within the manifold 130. In some embodiments, a region of the internal volume between the dispensing chambers 134a, 134b and the outlet port 136 can serve as a mixing chamber. Alternatively, in some embodiments, a longitudinally-extending partition separating the dispensing chambers 134a, 134b can extend only partly along a length of the dispensing chambers 134a, 134b, such that bottom end portions of the chambers 134a, 134b are in fluid communication and can provide initial mixing prior to the outlet port 136. Alternatively, in some embodiments, the dispensing chambers 134a, 134b are in fluid communication along their entire longitudinal length (e.g., no separating partition), such that the dispensing chambers 134a, 134b in combination serve as a mixing chamber.

The manifold 130 can further include a pair of longitudinally-extending piercing elements 132a, 132b, which can be an integrated part of a sidewall of the manifold 130 defining the dispensing chambers 134a, 134b, or be coupled to the manifold 130 to extend from an internal sidewall thereof. The piercing elements 132a, 132b can be substantially aligned with the fluid seals 124a, 124b of the BFS module 110. In the first configuration 100 of FIG. 1A, the piercing elements can be spaced from, or in non-piercing contact with fluid seals 124a, 124b, such that the liquid agents remain sealed within the respective reservoirs 120a, 120b. In some embodiments, the dispensing chambers 134a, 134b can have a funneled portion connecting to a top end of the outlet port 136. For example, the funneled portion can have a frustoconical or tapered shape in cross-section that narrows toward the outlet port 136. In some embodiments, each piercing element 132a, 132b is disposed on or extends from an inclined internal sidewall of the manifold 130 that defines the tapered shape of the funneled portion.

According to some embodiments, the manifold 130 can be coupled with the BFS module 110 via application of a longitudinal mating force. The BFS module 110 can be urged into an open longitudinal end of the manifold 130, for example, such that cooperatively-shaped interior chambers and/or grooves (e.g., scalloped sidewalls, not shown) accept the neck portions 112a, 112b (e.g., with cooperating surface features, such as a toroidal or doughnut shaped protrusion), thereby removably coupling the BFS module 110 to the manifold 130. In some embodiments, the interior chambers and/or grooves (and/or other interior features) and/or the neck portions 112a, 112b may be shaped such that uncoupling of the BFS module 110 from the manifold 130 is mechanically prohibited, or at least resisted. According to some embodiments, insertion of the neck portions 112a, 112b into the manifold 130 and/or otherwise mating thereof may cause the reservoirs 120a, 120b to remain outside of and/or axially adjacent to the manifold 130 (e.g., exposed from the manifold).

In some embodiments, the medical delivery assembly comprises an administration assembly, which can include an administration member 156 coupled to an administration hub 150. In some embodiments, the administration hub 150 can be coupled to (e.g., screwed onto) or integrated with the manifold 130 (e.g., adjacent outlet port 136). For example, the hub 150 can comprise internal threads that correspond and cooperate with external threads of the manifold 130, such that they may be rotationally and/or removably coupled together. In some embodiments, the administration member 156 can be inserted into and/or through the hub 150, for example, such that it comprises a first or administration end extending longitudinally distal from the BFS module 110 and a second end disposed within the hub 150 and/or extending into the manifold (e.g., in the case that the manifold 130 is coupled to the hub 150). In some embodiments, the administration end and/or a distal portion of the administration member 156 may be housed, shrouded, and/or covered by a cap (not shown), which may be removably coupled to the hub 150 (e.g., by fitting over an external portion thereof).

According to some embodiments, the manifold 130 and hub 150 combination may be utilized to couple and/or mate the administration member 156 with the BFS module 110 to provide a mechanism by which the administration member 156 may be coupled to the soft plastic BFS module 110 in a reliable manner. Due to the nature of the BFS plastic and/or process and/or the small form-factor of the BFS module 110, for example, providing external threads directly on the BFS module 110 would not be a viable option, as it may result in an imprecise, unreliable, and/or non-water tight coupling (e.g., the threads would be deformable even if they could be properly manufactured to within the desired tolerances, which itself is not a likely result) between the BFS module 110 and the hub 150. Applicant has realized, for example, that the “soft” plastics required for BFS process are not susceptible to machining due to heat deformation of machined features during formation attempts as well as deformation due to mechanical stress during utilization.

In some embodiments, the administration member 156 may comprise a needle for at least one of subcutaneous, intramuscular, intradermal, and intravenous injection of the fluid agent into the patient. For example, the needle can have a length of 0.5 mm to 4 mm, inclusive, or in a range of 4 mm to 15 mm, inclusive, or in a range of 15 mm to 30 mm, inclusive, depending on the desired manner of injection. For ease of explanation and description, the figures and the description herein generally refer to the administration member as a needle. However, it should be noted that, in other embodiments, the administration member 106 may include a nozzle (not shown) configured to control administration of the fluid agent to the patient. The nozzle may include a spray nozzle, for example, configured to facilitate dispersion of the fluid agent into a spray. Accordingly, a hub 104 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a hub 104 including a droplet nozzle may be useful in the administration of a fluid agent by way of droplets, such as administration to the eyes, topical administration, and the like.

In some embodiments, the medical delivery assembly comprises a removable casing 160. In some embodiments, the casing 160 can be constructed to (i) slide over the BFS module 110 and seat or couple with the manifold 130 (e.g., in a first orientation) and/or (ii) slide forcibly over and compress the fluid reservoirs 120a, 120b (e.g., simultaneously, upon application of a longitudinal force thereto; e.g., in a second and/or opposite orientation). For example, the casing 160 can comprise and/or define a first chamber portion 162a that extends from a first longitudinal end 160a, and a second chamber portion 162b that extend from a second longitudinal end 160b. In a first orientation, the first chamber portion 162a (e.g., an internal protection chamber) can receive therein a part of the BFS module 110 exposed from the manifold 130, as shown in FIG. 1A. For example, the first chamber portion 162a can be sized and shaped to accommodate therein the reservoirs 120a, 120b of the BFS module 110 without contacting, or at least without compressing, the reservoirs 120a, 120b. In some embodiments, a shape or contours of the chamber portion 162a (e.g., in a cross-section parallel to the lateral direction) can match a shape or contours of the reservoirs 120a, 120b (e.g., in the same cross-section).

In a second orientation, the second chamber portion 162b (e.g., an internal actuation chamber) can receive therein a part of the BFS module 110 exposed from the manifold, as shown in FIGS. 1B-1D. For example, the second chamber portion 162b can have an area in a cross-section parallel to the lateral direction that is less than that of the internal actuation chamber of the removable casing. In some embodiments, the second chamber portion 162b can have a rectangular shape in lateral cross-section (e.g., in a plane parallel to the lateral direction), which may remain substantially constant (e.g., in area and location) along the longitudinal direction of the casing 160. In some embodiments, first and second chambers portions 162a, 162b are opposite end portions of a continuous chamber extending between the first and second longitudinal ends 160a, 160b. Alternatively, in some embodiments, first and second chamber portions 162a, 162b are unconnected chambers (e.g., separated by an intervening solid partition). In some embodiments, instead of single casing, the first and second chamber portions 162a, 162b are provided in separate interchangeable casings, where a first casing with first chamber portion 162a is used to protect the BFS module 110 until administration is desired, and a second casing with second chamber portion 162b is used to activate the BFS module 110 (e.g., by breaching the fluid seals) and/or dispense the fluid agents (e.g., by compressing the reservoirs 120a, 120b) after removal of the first casing.

To transition the assembly from the first configuration 100a of FIG. 1A to the second configuration 100b of FIG. 1B, the casing 160 in the first orientation (e.g., with the first chamber portion 162a facing toward the manifold 130) can be removed from the BFS module 110, and flipped to the second orientation (e.g., with the second chamber portion 162b facing toward the manifold 130). The second chamber portion 162b of the casing 160 can then be pushed longitudinally onto the upper end of the BFS module 110, which in turn pushes pushing the BFS module 110 further into the manifold 130. The BFS module is thus forced into contact with the piercing elements 132a, 132b, such that piercing elements breach the fluid seals 124a, 124b of the liquid-agent-containing reservoirs 120a, 120b, as shown in FIG. 1B. Further application of longitudinal force to the casing 160 can further push the end of the BFS module 110 into the second chamber portion 162b, such that the second chamber portion 162b begins to compress the reservoirs 120a, 120b, thereby forcing the contents out of the reservoirs 120a, 120b. For example, the fluid agents from each reservoir 120a, 120b can flow around the piercing elements 132a, 132b extending through the seals 124a, 124b and into the dispensing chambers 134a, 134b (e.g., funneled portion) and subsequently to outlet port 136 of the manifold 130. The dispensed fluid agents mix together within the manifold to form a desired combination for a fluid or drug agent to be administered, as shown by the third configuration 100c of FIG. 1C. After mixing, the assembly can be converted to the fourth configuration 100d of FIG. 1D for administration of the combined fluid agents to a patient, for example, by further displacing the casing 160 over the part of the BFS module 110 exposed from the manifold 130, such that the reservoirs 120a, 120b are further compressed (e.g., mostly or fully compressed), thereby forcing the combined fluids from the outlet port 136 into the administration member 156 for delivery to a patient at 102.

As generally understood, the fluid or drug agent may include any type of agent to be injected into a patient (e.g., mammal, either human or non-human, or any other animal) and capable of producing an effect (alone, or in combination with an active ingredient). Accordingly, the agent may include, but is not limited to, a vaccine, a drug, a therapeutic agent, a medicament, a diluent, and/or the like. According to some embodiments, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components of the pre-filled multi-liquid medical delivery assembly, such as the BFS module 110, the hub 150, the manifold 130, and/or the casing 160.

According to some embodiments, the manifold 130, the hub 150, and/or the casing 160 may be composed of a medical grade material. In some embodiments, the manifold 130, the hub 150, the casing 160, and/or the cap for the administration member 156, may be composed of a thermoplastic polymer or other relatively hard plastic (e.g., greater than 80 on the Rockwell “M” scale; e.g., Rockwell M 85; and/or greater than 110 on the Rockwell “M” scale; e.g., Rockwell R 115), such as, but not limited to, polypropylene, polybenzimidazole, acrylonitrile butadiene styrene (ABS), polystyrene, polyvinyl chloride, polycarbonate, or the like. According to some embodiments, the manifold 130, the hub 150, and/or the casing 160 may be constructed to have an ultimate tensile strength of greater than eighty megapascals (>80 MPa), for example, approximately eighty-two and seven tenths megapascals (82.7 MPa). In some embodiments, at least the casing 160 is formed of a material having a hardness greater than that of the manifold 130. For example, the casing can be formed of polypropylene (e.g., high-density polypropylene), the manifold can be formed of polycarbonate, and the BFS module can be formed of polyethylene (e.g., low-density polyethylene (LDPE)).

In some embodiments, the pre-filled multi-liquid medical delivery assembly may be advantageously manufactured (in mass quantities) in separate parts or portions, namely, at least the relatively soft plastic BFS module 110 portion (e.g., a “first” piece), the relatively hard plastic manifold (e.g., a “second” piece), the hub 150, administration member 156, and corresponding cap for the administration member (e.g., a “third” piece), and/or the casing 160 (e.g., a “fourth” piece) with such different plastic parts/portions being selectively coupled to administer a medication (e.g., of at least two fluids) to a patient. In some embodiments, the BFS module 110 may be specially designed and/or configured to separately store or house the at least two different fluids until such time as the pre-filled multi-liquid medical delivery assembly is activated (e.g., by breaching the sealed ports, dispensing the fluid agents from the BFS vial into the manifold, and subsequently injecting the mixed fluid agents into the patient).

In some embodiments, the pre-filled medical delivery assembly or system may comprise various inter-connected and/or modular components. For example, the pre-filled multi-liquid medical delivery assembly may include a modular design consisting of separately constructed components 110, 130, 150, 156, and 160, cooperatively arranged and coupled to one another.

In some embodiments, some or all of the various components can be provided in an unassembled state as a kit, for example, for assembly in the field and/or at a time just prior to administration. In some embodiments, the pre-filled medical delivery assembly, or the kit for forming the medical delivery assembly, can include additional components beyond those specifically illustrated in FIGS. 1A-1D. In some embodiments, the components of the kit, e.g., the BFS module 110, the manifold 130, the administration assembly (e.g., hub 150 and member 156), and/or the casing 160 can be manufactured, packaged, shipped, stored, and/or provided as separate components. In such a manner, the manifold, administration assembly, and/or casing may not need to be stored or shipped in accordance with often restrictive requirements imposed on medicaments and may accordingly reduce the amount of space required for such specialized storage and/or shipping. The manifold, the casing, and/or the administration assembly may also or alternatively be manufactured, stored, and/or shipped in advance (e.g., at a first time) while the BFS module 110 that is pre-filled with the liquid agents may be manufactured, stored, and/or shipped at a later time (e.g., a second time). In some embodiments, the delay between the first time and the second time may be lengthy without causing detrimental effects, as the manifold, the casing, and/or the administration assembly may be stored, in some embodiments, indefinitely. In such a manner, units of the manifold, the casing, and/or the administration assembly may be provided to be on-hand in advance of the availability and/or arrival of the BFS module 110, reducing supply chain constraints in the case of proactive mixing assembly procurement. Alternatively or additionally, at least some components of the assembly may be reusable with other BFS modules, for example, the casing 160. Thus, unassembled components of pre-filled medical delivery system kits according to embodiments of the disclosed subject matter need not be provided together in the same package or at the same time.

In some embodiments, fewer or more components 102-162 and/or various configurations of the depicted components 102-162 may be included in the pre-filled multi-liquid medical delivery assembly without deviating from the scope of embodiments described herein. In some embodiments, the components 102-162 may be similar in configuration and/or functionality to similarly named and/or numbered components as described herein.

### II. Multi-Fluid Blow-Fill-Seal (BFS) Modules

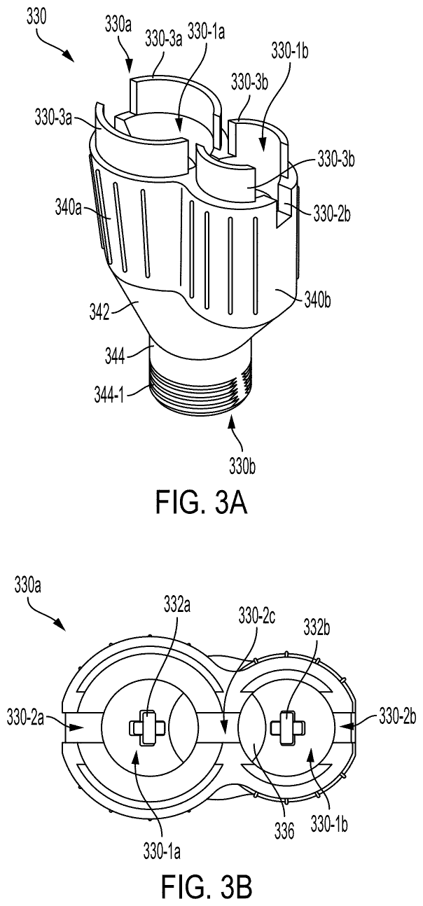

Referring to FIGS. 2A-2C, a multi-fluid BFS module 310 according to some embodiments is shown. The BFS module 310 can have a first longitudinal end 310a (e.g., a coupling end) and a second longitudinal end 310b (e.g., a free end). In some embodiments, the multi-fluid BFS module 310 may comprise a plurality of neck portions 312a-b at or proximal to the first longitudinal end 310a. In some embodiments, each neck portion 312a-b can comprise an external coupling or mating feature 314a-b (e.g., a radially-protruding portion, forming a toroidal or doughnut shape in a side view). The BFS module 310 may also comprise a plurality of fluid reservoirs 320a-b, which can be joined together via a central web 316. Alternatively or additionally, the fluid reservoirs 320a-b can comprise and/or be coupled to side ribs, flanges, or webs 318a-b (e.g., that extend longitudinally along opposing side portions of the multi-fluid BFS module 310). In the illustrated example, each of the fluid reservoirs 320a-b may be sized and/or configured to define different interior volumes for different fluid agents (not separately shown) stored therein.

In some embodiments, each neck portion 312a-b is in fluid communication and axially aligned with a respective fluid reservoir 320a-b. In some embodiments, each neck portion 312a-b may be sealed via a respective fluid seal 324a-b at a terminal end thereof. In some embodiments, the seals 32a-b comprise a foil, wax, paper, a section of the BFS module (e.g., a bottom wall of the BFS module), or any combination of the foregoing. In some embodiments, the seals 324a, 324b can be breached, for example, by puncturing, piercing, rupturing, penetrating, or otherwise breaking the seal, in order to access and/or dispense contents of the fluid reservoirs 320a, 320b.

In some embodiments, the multi-fluid BFS module 310 may be constructed (e.g., formed) via a BFS process and may comprise a “soft” plastic (e.g., having a Shore/Durometer “OO” hardness of between 60 and 70 and/or a Shore/Durometer “A” hardness between 20 and 50) that is not functionally susceptible to the formation and/or utilization of threaded connection features (as are possible to form on different, harder plastics). In some embodiments, the BFS module 310 can have a length, L₁, along the longitudinal direction of about 49.9 mm, a first width, W₁, along the lateral direction of about 20.7 mm, and a second width, W₂, in a direction perpendicular to the longitudinal and lateral directions of about 10 mm.

In some embodiments, fewer or more components 312-324 and/or various configurations of the depicted components 312-324 may be included in a multi-fluid BFS module without deviating from the scope of embodiments described herein. In some embodiments, the components 312-324 may be similar in configuration and/or functionality to similarly named and/or numbered components as described herein.

### III. Manifolds for Pre-Filled Multi-Fluid Medical Delivery Assemblies

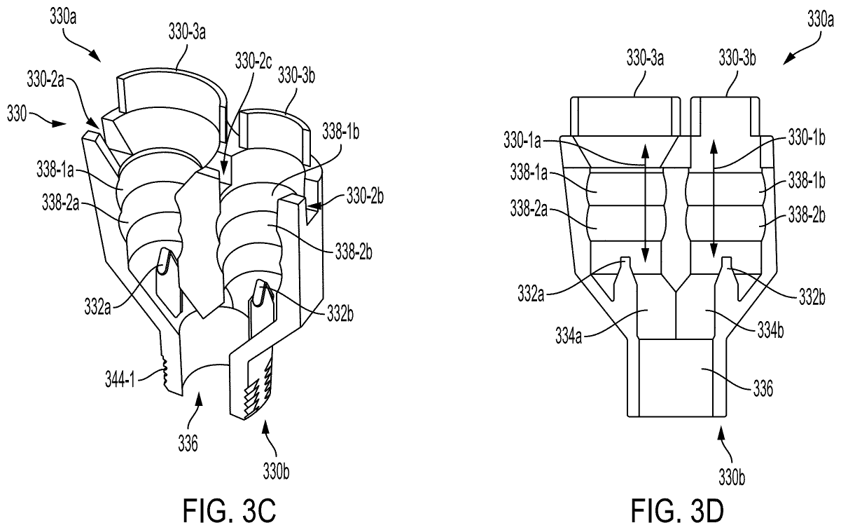

Referring to FIGS. 3A-3D, a manifold 330 (also referred to herein as multi-port BFS coupling) according to some embodiments is shown. According to some embodiments, the manifold 330 can comprise a hard plastic molded modular component of a pre-filled multi-liquid medical delivery assembly that permits a multi-chamber BFS module to be utilized to inject a single dose of a multi-fluid agent into a patient. In some embodiments, manifold 330 can comprise and/or define, for example, a plurality of inlet ports or axial bores 330-1a, 330-1b and/or one or more lateral or radial grooves, tracks, and/or slits 330-2a, 330-2b, 330-2c. In some embodiments, the manifold 330 can comprise one or more axial mounting guides and/or features 330-3a, 330-3b. According to some embodiments, each of the axial bores 330-1a, 330-1b, the lateral slits 330-2a, 330-2b, 330-2c, and/or the mounting features 330-3a, 330-3b may be disposed at a first end 330a of the manifold 330.

According to some embodiments, the axial bores 330-1a, 330-1b can be sized and/or shaped to receive portions (e.g., necks) of a BFS module, such as necks 312a, 312b of BFS module 310 of FIGS. 2A-2C. In the illustrated example, a first axial bore 330-1a is shaped to accept a cylindrically-shaped element having a first diameter and a second axial bore 330-1b is shaped to accept a cylindrically-shaped element having a second diameter (e.g., smaller than the first diameter). In some embodiments, the lateral slits 330-2a, 330-2b, 330-2c may be sized and/or shaped to permit one or more flanges, wings, and/or projections or other features (not shown) of a BFS module to be seated therein, such as side ribs 318a, 318b and central web 316 of BFS module 310 of FIGS. 2A-2C. In such a manner, for example, an engagement of a BFS module having such features engaged in or with one or more of the lateral slits 330-2a, 330-2b, 330-2c can be restrained from axial rotation with respect to the manifold 330.

In some embodiments, the manifold 330 can comprise piercing elements 332a-b, each aligned with a respective one of the axial bores 330-1a, 330-1b and/or disposed in a respective one of the dispensing chambers 334a, 334b. As shown in FIGS. 3B-3C, the piercing elements 332a-b may, in some embodiments, comprise an “X” and/or cross-shaped configuration (e.g., with one cross-member protruding higher than the other) and/or may be integral to the manifold 330. In some embodiments, piercing elements 332a-b may be constructed of a different material than the manifold 330 and/or may be coupled to the manifold 30. In some embodiments, the dispensing chambers 334a-b may meet or join at and/or be in fluid communication with an outlet port 336. According to some embodiments, and as shown in FIGS. 3C-3D, the multi-port BFS coupling 330 may comprise a plurality of mating and/or indexing features 338-1a, 338-1b, 338-2a, 338-2b. In some embodiments, the mating features 338-1a, 338-1b, 338-2a, 338-2b may be disposed in each of the axial bores 330-1a, 330-1b. In the illustrated example, each axial bore 330-1a, 330-1b may comprise a first mating feature 338-1a, 338-1b, such as a rounded interior groove or radial seat (e.g., a scalloped sidewall), disposed axially adjacent to a lower axial terminus of the lateral slits 330-2a, 330-2b, 330-2c.

In some embodiments, each axial bore 330-1a, 330-1b may comprise a second mating feature 338-2a, 338-2b, such as a rounded interior groove or radial seat (e.g., a scalloped sidewall), disposed axially adjacent to a lower axial terminus of the first mating features 338-1a, 338-1b. According to some embodiments, the second mating features 338-2a, 338-2b may be axially distanced from the tips of the respective piercing elements 332a-b by a distance designed to cause the piercing elements 332a-b to pierce a seal of the BFS module in the case that a mounting flange (e.g., the coupling feature 314 of the neck portion 312 of the BFS module 310) is seated in the second mating features 338-2a, 338-2b. In some embodiments, the mating features 338-1a, 338-1b, 338-2a, 338-2b may comprise any number or configuration of shaped features that permit selective, indexed, removable, and/or non-removable coupling between the manifold 330 and the BFS module.

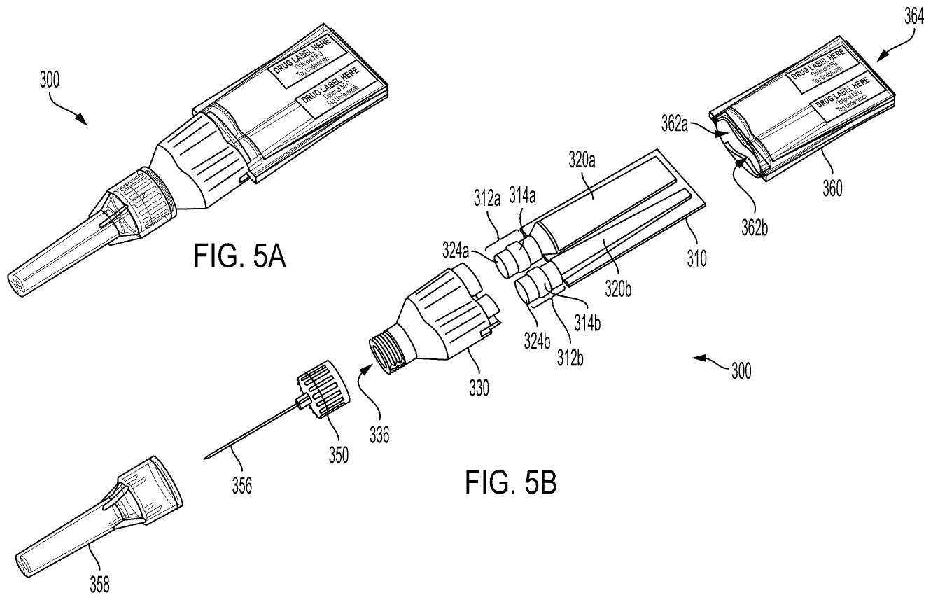

According to some embodiments, the manifold 330 may generally comprise two interconnected and/or jointly formed tubes 340a-b (e.g., cylindrical tubes) coupled to and/or formed with a mixing body 342 and/or an outlet tube 344. The tubes 340a-b may comprise and/or define the axial bores 330-1a, 330-1b. For example, the mixing body 342 may comprise and/or define the outlet chambers 334a-b, and/or the outlet tube 344 may comprise and/or define the outlet port 336. In some embodiments, the outlet tube 344 may be configured to couple to one or more other objects or modules at the second end 330b of the manifold, such as a needle hub (e.g., needle hub 350 in FIGS. 5A-5B), for example, via external threads 344-1 as shown. For example, interior threads of the needle hub may correspond to and/or mate with the external threads 344-1 of the neck of the manifold 330, so as to permit selective coupling therebetween.

In some embodiments, fewer or more components 330-1a-344-1 and/or various configurations of the depicted components 330-1a-344-1 may be included in the manifold 330 without deviating from the scope of embodiments described herein. In some embodiments, the components 330-1a-344-1 may be similar in configuration and/or functionality to similarly named and/or numbered components as described herein.

### IV. Casings for Pre-Filled Multi-Fluid Medical Delivery Assemblies

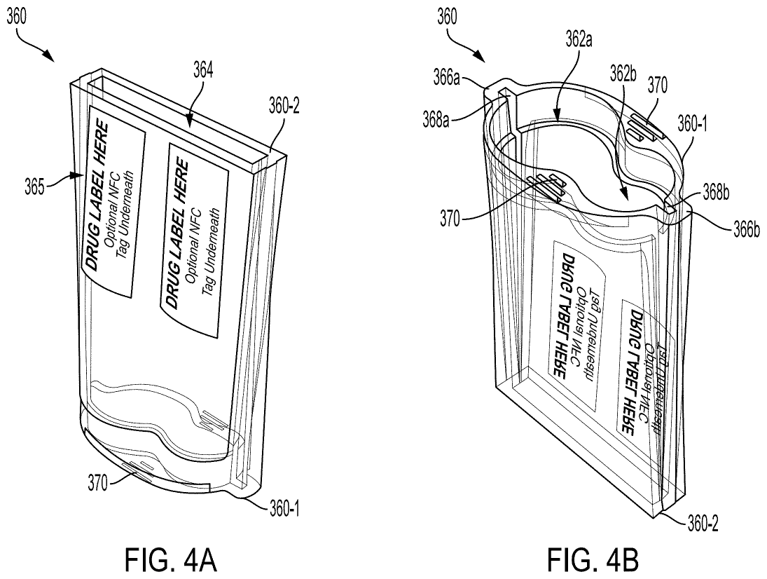

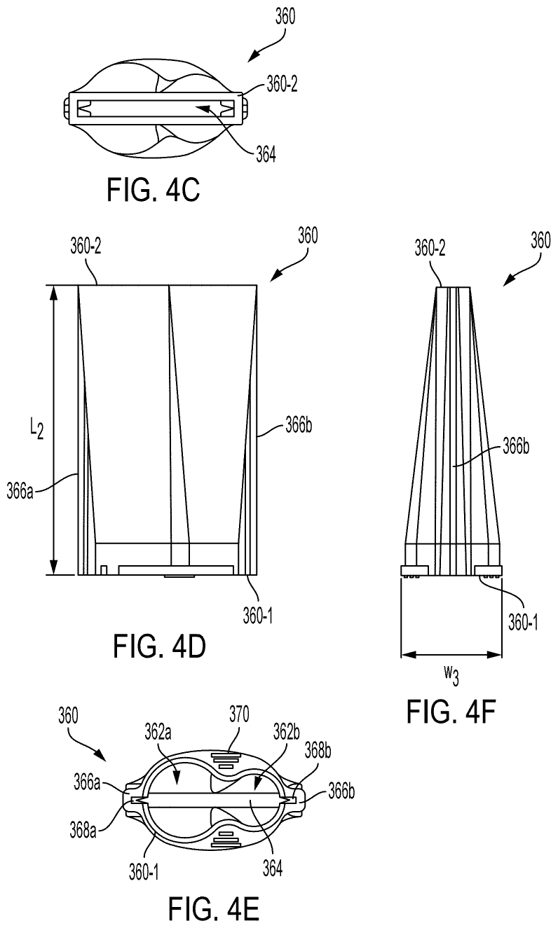

Referring to FIGS. 4A-4F, a casing 360 (also referred to herein as housing) according to some embodiments is shown. According to some embodiments, the casing 360 may comprise a modular component of a pre-filled multi-liquid medical delivery assembly that permits a multi-chamber BFS module to be utilized to inject a single dose of a multi-fluid agent into a patient. In some embodiments, casing 360 may be constructed of hard and/or durable plastic or other materials, for example, and may shield and/or protect the BFS module 310. In some embodiments, the casing 360 may releasably couple to the manifold 330, for example, at first longitudinal end 330 (e.g., over or in contact with axial mounting guide portions 330-3a, 330-3b). In some embodiments, the casing 360 may comprise and/or define, for example, a first longitudinal end 360-1 and a second longitudinal end 360-2, each having different profiles and/or shapes. In some embodiments, the first longitudinal end 360-1 may comprise and/or define a first shaped chamber 362a-b (e.g., having a first cross-sectional profile) while the second longitudinal end 360-2 may comprise and/or define a second shaped chamber 364 (e.g., having a second cross-sectional profile). According to some embodiments, the first shaped chamber 362a-b may comprise and/or define a first cylindrical chamber portion 362a having a first diameter and a second cylindrical chamber portion 362b having a second diameter. In some embodiments, the first and second cylindrical chamber portions 362a-b may be joined and/or in communication, e.g., defining the single first shaped chamber 362a-b. The first shaped chamber 362a-b may be sized and/or shaped, for example, to accept a BFS module (e.g., such as BFS module 310)—e.g., to shield the BFS module, without application of pressure to any fluid reservoirs thereof.

According to some embodiments, the second shaped chamber 364 may comprise and/or define a rectangular profile that may be sized and/or shaped, for example, to accept the BFS module in its non-filled state (e.g., not sized to accept any filled fluid reservoirs thereof without requiring an application of pressure to such fluid reservoirs thereof). The casing 360 may, for example, comprise multi-use modular components that protect the BFS module in a first orientation (e.g., in the case that the BFS module is inserted into the first shaped chamber 362a-b) and may also be utilized to compress and/or expel fluids from the BFS module in the case it is engaged in a second orientation (e.g., in the case that the BFS module is forcibly inserted into the second shaped chamber 364).

In some embodiments, the casing 360 may comprise and/or define one or more longitudinal flanges 366 that house and/or define corresponding internal axial slits, grooves, and/or tracks 368. The internal axial tracks 368 may be sized and/or shaped, for example, to accept one or more exterior flanges, wings, webs, and/or other projections of the BFS module. In such a manner, for example, insertion of the BFS module into the casing 360 may be facilitated by alignment of the interior axial tracks 368 with the corresponding features of the BFS module. According to some embodiments, the casing 360 may comprise one or more grip or push surfaces 370. In the illustrated example, two opposing and radially-projecting (e.g., perpendicular to a longitudinal axis of the casing 360) push surfaces 370 may be formed, coupled, and/or otherwise disposed at the first end 360-1. According to some embodiments, these push surfaces 370 may provide areas that a user can utilize to apply axial and/or longitudinal force to the casing 360. In the case that the second end 360-2 of the casing 360 is forced onto the BFS module (and/or the BFS module is forced into the second shaped chamber 364), for example, the user may apply force to urge the casing 360 onto the BFS module (e.g., thereby compressing any fluid reservoirs protruding radially or laterally therefrom).

In some embodiments, fewer or more components 362a-370 and/or various configurations of the depicted components 362a-370 may be included in the casing 360 without deviating from the scope of embodiments described herein. In some embodiments, the components 362a-370 may be similar in configuration and/or functionality to similarly named and/or numbered components as described herein.

### V. Pre-Filled Multi-Fluid Medical Delivery Assemblies

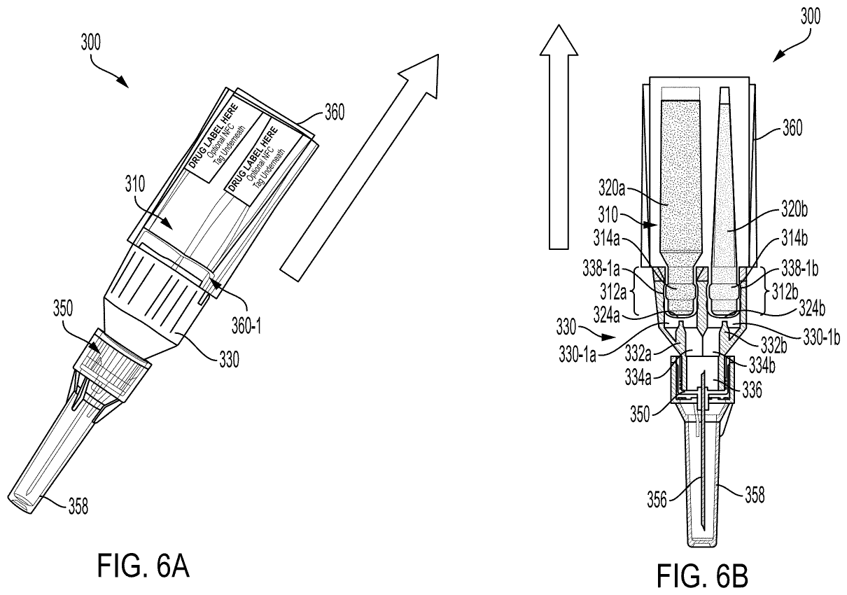

Referring to FIGS. 5A-5B and 6A-6B, a pre-filled multi-liquid medical delivery assembly 300 is shown. The pre-filled multi-liquid medical delivery assembly 300 may utilize and/or employ a BFS module 310 to safely, inexpensively, reliably, and/or conveniently administer a multi-fluid medicament to a patient or other target (not shown). The BFS module 310 may comprise, for example, a plurality of neck portions 312a-b, each neck portion 312a-b comprising a soft-plastic mating feature such as an exterior rounded flange or “doughnut” 314a-b. According to some embodiments, the neck portions 312a-b may define two or more adjacently disposed portions of the BFS module 310 that are connected via a web or bridge 316 and/or that define or are coupled to lateral flanges, ribs, or wings 318. In some embodiments, each of the two portions of the BFS module 310 may comprise and/or define a fluid reservoir 320a-b that defines a respective reservoir volume (not separately labeled) that is in communication with the respective and/or aligned neck portion 312a-b. In some embodiments, each connected neck portion 312a-b and fluid reservoir 320a-b pair may house and/or contain a combination of liquid and gaseous components. When disposed in a “downward” configuration as depicted in FIG. 6B, for example, the liquid components may rest in the respective neck portions 312a-b and partially within the respective fluid reservoirs 320a-b (e.g., substantially or entirely in the reservoir volumes) against respective fluid seals 324a-b thereof and any gaseous components may be substantially disposed in the respective fluid reservoir volumes. In some embodiments, a first fluid or liquid in a first fluid reservoir 320a may comprise approximately two milliliter (2.0 ml) of a first fluid/mixture and/or a second fluid or liquid in a second fluid reservoir 320b may comprise approximately seven tenths of a milliliter (0.7 ml) of second fluid/mixture (e.g., that when combined, form an injectable medicament or other combined agent).

According to some embodiments, the pre-filled multi-liquid medical delivery assembly 300 may comprise a mixing connector or manifold 330 that comprises and/or defines one or more chambers 330-1a, 330-1b configured to receive and/or mate with one or more of the necks 312a-b and/or fluid reservoirs 320a-b. In some embodiments, and as depicted in a first state or configuration in FIGS. 5A and 6A-6B, the fluid reservoirs 320a-b and respective necks 312a-b may be disposed in the respective chambers 330-1a, 330-1b of the manifold 330. According to some embodiments, the manifold 330 may comprise mating slots 330-2 configured (e.g., sized and positioned) to receive and/or couple to the wings 318 of the BFS module 310 (e.g., in the case that the BFS module 310 is engaged with the manifold 330, as depicted in FIGS. 5A and 6A-6B). In some embodiments, the manifold 330 may comprise one or more axial mounting flanges 330-3a, 330-3b. In some embodiments, the manifold 330 may comprise a plurality of piercing elements 332a-b disposed adjacent to the respective fluid seals 324a-b of the necks 312a-b of the BFS vial 310. In a first state of insertion of the BFS vial 310 into the manifold 330 as depicted in FIGS. 5A and 6A-6B, for example, the fluid seals 324a-b may be disposed adjacent and/or proximate to, or even rest upon the tips of the piercing elements 332a-b (e.g., without breaching the integrity of the seals). In some embodiments, the manifold 330 may comprise a dispensing chamber 334a-b for each respective neck 312a-b/reservoir 320a-b. According to some embodiments, one or more of the piercing elements 332a-b may be disposed within each of the dispensing chambers 334a-b.

In some embodiments, the manifold 330 may comprise and/or define an outlet port 336 in fluid communication with the dispensing chambers 334a-b. The outlet port 336 may be disposed, for example, in a neck portion of the manifold 330 that comprises external threads thereon. In some embodiments, the manifold 330 may comprise, inside each of the respective chambers 330-1a, 330-1b, a plurality of mating and/or indexing features 338-1a, 338-1b, 338-2a, 338-2b. In the first state of insertion of FIGS. 5A and 6A-6B, for example, the exterior rounded flanges 314a-b of the necks 312a-b of the BFS vial 310 may be engaged to be seated within respective first mating features 338-1a, 338-1b. According to some embodiments, the mating features 338-1a, 338-1b, 338-2a, 338-2b may comprise one or more interior grooves, slots, recesses, indents, tracks, apertures, etc., sized and/or positioned to selectively couple to and/or index with the exterior rounded flanges 314a-b of the necks 312a-b of the BFS vial 310. In some embodiments, the exterior rounded flanges 314a-b of the necks 312a-b of the BFS vial 310 and the mating features 338-1a, 338-1b, 338-2a, 338-2b may be cooperatively shaped to permit the BFS vial 310 to be snapped or “clicked” into the manifold 330 in the first state or position of FIGS. 6A-6B.

According to some embodiments, a needle hub 350 may be coupled to the manifold 330. Interior threads of the needle hub 350 may correspond to and/or mate with the external threads of the neck of the manifold 330, for example, permitting selective coupling therebetween. In some embodiments, the needle hub 350 may comprise a needle 356 (and/or other and administration member) and/or a cap 358 that protects and/or shields at least a distal end of the needle 356.

According to some embodiments, the pre-filled multi-liquid medical delivery assembly 300 may comprise a removable casing 360. The removable casing 360 may be constructed of hard and/or durable plastic or other materials, for example, and may shield and/or protect the BFS module 310 (and/or the manifold 330 and/or needle hub 350). In some embodiments, the casing 360 may selectively couple to the manifold 330 and/or the BFS module 310. In a first orientation of the casing 360 as depicted in FIGS. 5A-6B (e.g., in a shipped or transport state), for example, a first end 360-1 of the casing 360 may be removably coupled to the manifold 330 such that the casing 360 covers, protects, and/or shields the BFS module 310. In some embodiments, this first state may comprise an assembled consumer and/or end-user (e.g., healthcare worker) state of the pre-filled multi-liquid medical delivery assembly 300 that represents a configuration in which it is provided for end use. In some embodiments, the pre-filled multi-liquid medical delivery assembly 300 may be transitioned from this first state to a second state by removal of the casing 360 (e.g., via application of a longitudinal and/or axial separation force applied between the casing 360 and the manifold 330; e.g., as indicated by the linear arrows in FIGS. 6A-6B).

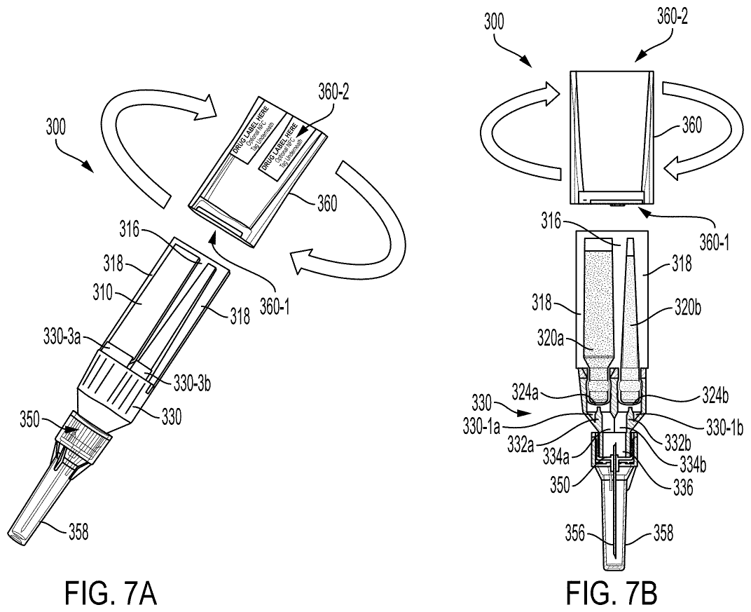

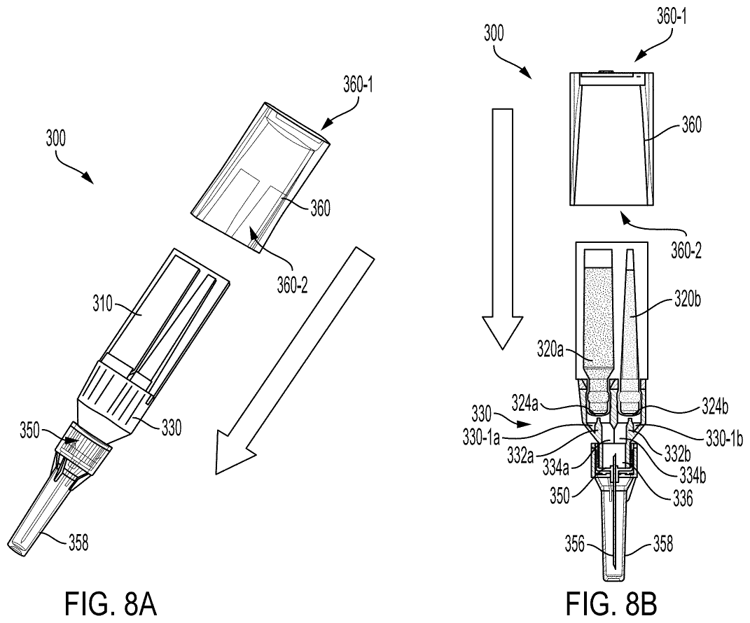

FIGS. 7A-7B illustrate an exemplary second state for the pre-filled multi-liquid medical delivery assembly 300. In the second state, the casing 360 has been separated from the manifold 330 (and/or from the BFS module 310), exposing the BFS module 310 (or at least any portions thereof that are not seated within the manifold 330). In some embodiments, the pre-filled multi-liquid medical delivery assembly 300 may be transitioned to a third state by an inversion or flipping of the casing 360 (e.g., as indicated by the rotational arrows in FIGS. 7A-7B, such that second longitudinal end 360-2 faces the manifold 330). FIGS. 8A-8B illustrate an exemplary third state for the pre-filled multi-liquid medical delivery assembly 300. In some embodiments, the pre-filled multi-liquid medical delivery assembly 300 may be transitioned to a fourth state by engagement of the inverted casing 360 with the BFS module 310 (e.g., as indicated by the linear arrows in FIGS. 8A-8B).

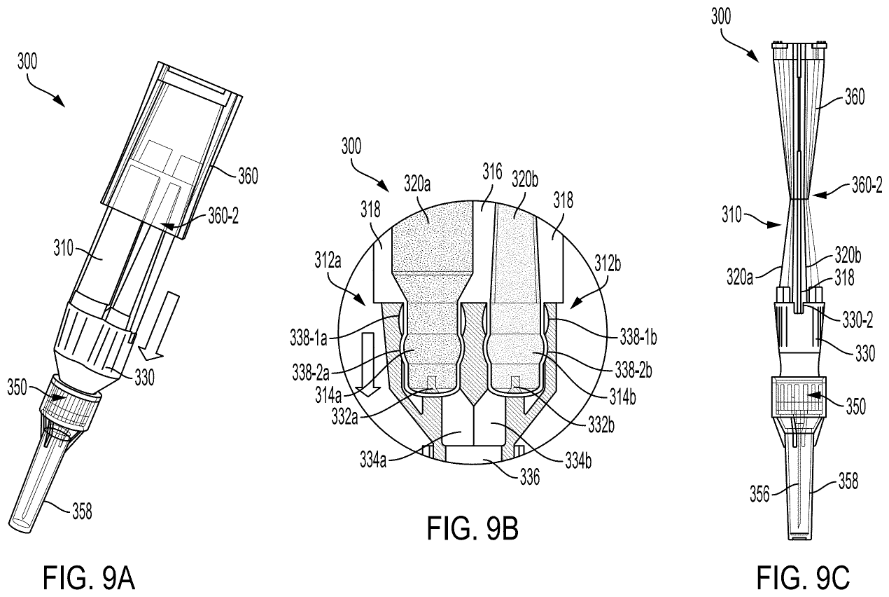

FIGS. 9A-9C illustrate an exemplary fourth state for the pre-filled multi-liquid medical delivery assembly 300. For example, the inverted casing 360 may be aligned such that a second end 360-2 thereof is axially oriented with the tail or back end of the BFS module 310 (e.g., free longitudinal end 310b) and the second end 360-2 of the casing 360 may be urged onto the BFS module 310. In the fourth state, the second end 360-2 of the casing 360 has been engaged with the BFS module 310 such that continued axial and/or longitudinal force is applied by the casing 360 to the BFS module 310. In some embodiments, this transfer of force may be due to the respective profiles and/or shapes of the BFS module 310 and the second end 360-2 of the casing 360. The first end 360-1 of the outer housing 360 may be shaped to fit over and/or house the BFS module 310 (e.g., by comprising and/or defining a cylindrical opening or openings that are shaped to accept the fluid reservoirs 320a-b). In contrast, the second end 360-2 may comprise a smaller and/or rectangular profile and/or shape that is configured to accept the wings 318 and the rib 316 of the BFS vial 310, but that is too small to accept the fluid reservoirs 320a-b in their fluid-filled or expanded-volume state (e.g., as well depicted in the side or profile view of FIG. 9C). As such, engagement of the second end 360-2 to fit over the BFS module 310 may cause the second end 360-2 to transfer the applied axial and/or longitudinal force to the BFS module 310 (e.g., as indicated by the linear arrow in FIGS. 9A-9B). In some embodiments, the transferred force may cause the BFS module 310 to advance to a second state of insertion into the manifold 330 (e.g., as depicted by the linear arrow in FIG. 9A and as well depicted in the zoomed-in partial cross section of FIG. 9B). In the second state of insertion, for example, the exterior rounded flanges 314a-b may become unseated from the first mating features 338a-1, 338-1b and advanced axially into respective second mating features 338-2a, 338-2b. According to some embodiments, advancement of the BFS module 310 to the second state of insertion into the manifold 330 may cause the piercing elements 332a-b to engage with and pierce, rupture, and/or otherwise break the respective fluid seals 324a-b. In some embodiments, the exterior rounded flanges 314a-b of the necks 312a-b of the BFS vial 310 and the second mating features 338-2a, 338-2b may be cooperatively shaped to permit the BFS vial 310 to be snapped or “clicked” into the manifold 330 in the second state or position of FIGS. 9A-9C.

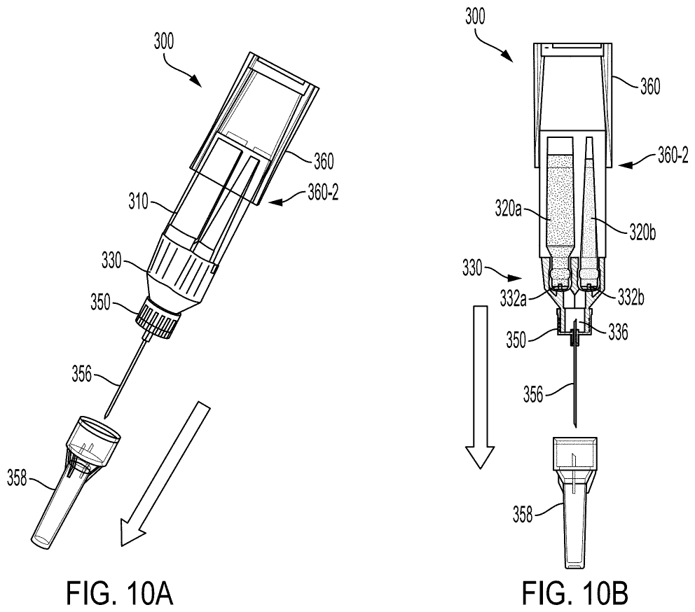

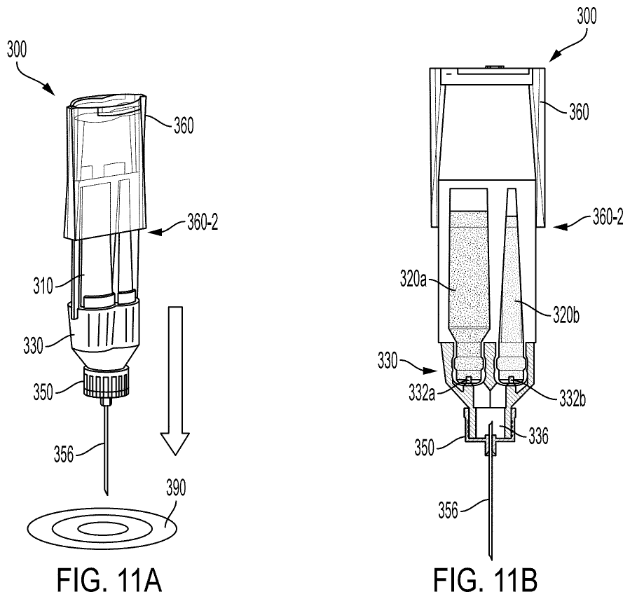

According to some embodiments, once the fluid seals 324a-b have been pierced, the pre-filled multi-liquid medical delivery assembly 300 may be advanced to a fifth state, for example, as depicted in FIGS. 10A-10B. In the fifth state, for example, the combined needle hub 350, manifold 330, BFS module 310, and inverted casing 360 may be prepared for injection/administration. As depicted in FIGS. 10A-10B, for example, the cap 358 may be removed to expose the administration end of the needle 356 (e.g., via application of an axial and/or longitudinal separation force applied between the manifold 330 and the cap 358; e.g., as indicated by the linear arrows in FIG. 10A-10B). In some embodiments, the pre-filled multi-liquid medical delivery assembly 300 may be advanced to a sixth state, for example, as depicted in FIGS. 11A-11B. In the sixth state, for example, the combined needle hub 350, manifold 330, BFS module 310, and inverted casing 360 may be positioned over a target 390 (e.g., a chosen administration site of a patient) and advanced toward the target (e.g., as indicated by the linear arrow in FIG. 11A).

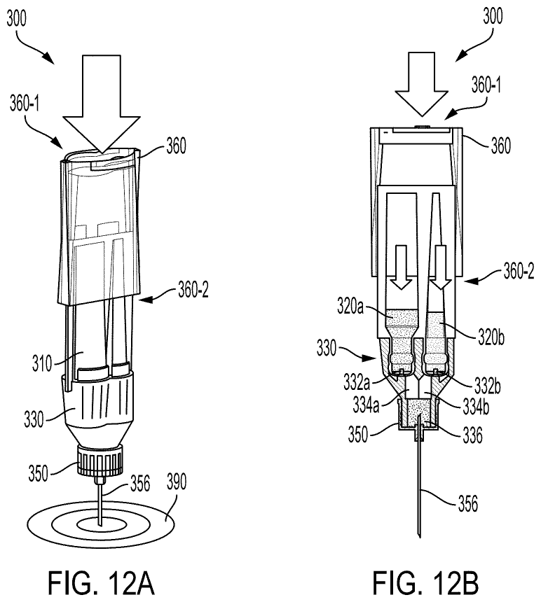

According to some embodiments, the pre-filled multi-liquid medical delivery assembly 300 may be advanced to a seventh state, for example, as depicted in FIGS. 12A-12B. In the seventh state, for example, the combined needle hub 350, manifold 330, BFS module 310, and inverted casing 360 may be engaged to administer the combined fluid agent/medicament to the patient. The needle 356 (and/or other administration member) may be inserted into the target 390, for example, and the inverted casing 360 may be advanced onto the BFS module 310 (via application of an axially downward force; e.g., as indicated by the linear arrows at the first end 360-1 of the inverted casing 360 in FIGS. 12A-12B). In some embodiments, advancement of the inverted outer housing 360 may cause the fluid reservoirs 320a-b to compress, thereby applying pressure to any gaseous components of the reservoirs 320a-b and thereby forcing the liquid components (at least partially) through the breaches in the fluid seals 324a and into the respective dispensing chambers 334a-b. According to some embodiments, the cross-sections of the piercing elements 332a-b may be configured to permit the fluids/liquids to enter the dispensing chambers 334a-b. The piercing elements 332a-b may, for example, comprise an “x” cross-section defining four (4) axial channels for fluid flow through the breaches. The dispensing chambers 334a-b may, in some embodiments, comprise a single mixing chamber that permits unfettered mixing and/or combining of the multiple fluid/liquid agents. In some embodiments, the dispensing chambers 334a-b may comprise various baffles, weirs, and/or other hydraulic features that partially separate the dispensing chambers 334a-b and/or provide for specific and/or enhanced mixing therein. According to some embodiments, as the dispensing chambers 334a-b may contain air or another gaseous component, the entering liquids/fluids may mix with such existing component(s) in the dispensing chambers 334a-b (and/or in the outlet port 336). In some embodiments, because the combined volumes of the reservoirs 320a-b, the necks 312a-b, and the dispensing chambers 334a-b (and/or in the outlet port 336) is smaller than in the previous states, one or more of the gaseous components may become at least partially compressed.

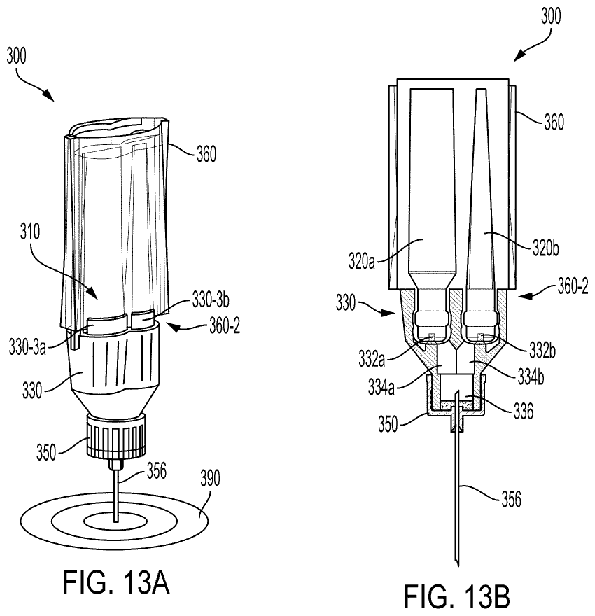

In some embodiments, the pre-filled multi-liquid medical delivery assembly 300 may be advanced to an eighth state, for example, as depicted in FIGS. 13A-13B. In the eighth state, for example, the inverted casing 360 may be further and/or fully advanced onto the BFS module 310 such that substantially all fluid agent from each fluid reservoir 320a-b is forced through the dispensing chambers 334a-b, into the outlet port 336, and through the needle 356 to administer the combined fluid agent/medicament to the patient. As depicted in FIGS. 13A-13B, the inverted casing 360 may be advanced axially along and/or over the BFS module 310 until the second end 360-2 engages with (e.g., is stopped by) the manifold 330. In some embodiments, the second end 360-2 may engage with and/or become seated against or with the axial mounting flanges 330-3a, 330-3b of the manifold 330, by which point all desired fluid agent (e.g., a desired dosage amount) will have been expelled from the fluid reservoirs 320a-b and into the patient. In some embodiments, some residual fluid agent(s) may remain in the manifold 330 (and/or the BFS module 310) in accordance with a designed dosage specification for the pre-filled multi-liquid medical delivery assembly 300.

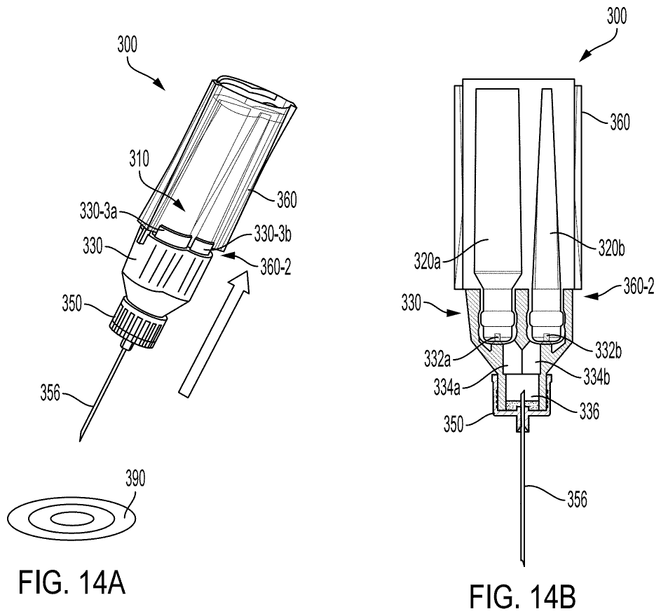

According to some embodiments, once the fluids/liquids have been introduced (e.g., at the eighth state) the pre-filled multi-liquid medical delivery assembly 300 may be advanced to a ninth state, for example, as depicted in FIGS. 14A-14B. In the ninth state, for example, the combined needle hub 350, manifold 330, BFS module 310, and fully-engaged inverted casing 360 may be removed from the patient, such as by backing the needle 356 out of the target site 390 (e.g., as indicated by the linear arrow in FIG. 14A). According to some embodiments, upon removal the pre-filled multi-liquid medical delivery assembly 300 may be empty, inert, and/or otherwise ready for disposal. In some embodiments (not depicted), the cap 358 may be reinstalled over the needle 356 to provide for a safer disposal.

According to some embodiments, the pre-filled multi-liquid medical delivery assembly 300 may include a modular design of separately constructed components 310, 330, 350, 356, and 360 cooperatively arranged and coupled to one another. Alternatively or additionally, in some embodiments one or more of the separately illustrated components can be combined together into an integrated component (e.g., formed together rather than formed separately and subsequently coupled together). For example, in some embodiments, the hub 350 may be an integrated part of manifold 330. In some embodiments, the casing 360 may be reusable with other BFS modules 310 and/or manifolds 330. In some embodiments, the BFS module 310 may be disposable, for example, after administration of combined liquid agents to the patient. In some embodiments, both the BFS module 310 and manifold 330 may be disposable.

In some embodiments, fewer or more components 310-370 and/or various configurations of the depicted components 310-370 may be included in the pre-filled multi-liquid medical delivery assembly 300 without deviating from the scope of embodiments described herein. In some embodiments, the components 310-370 may be similar in configuration and/or functionality to similarly named and/or numbered components as described herein.

### VI. Pre-Filled Multi-Fluid Medical Delivery Methods

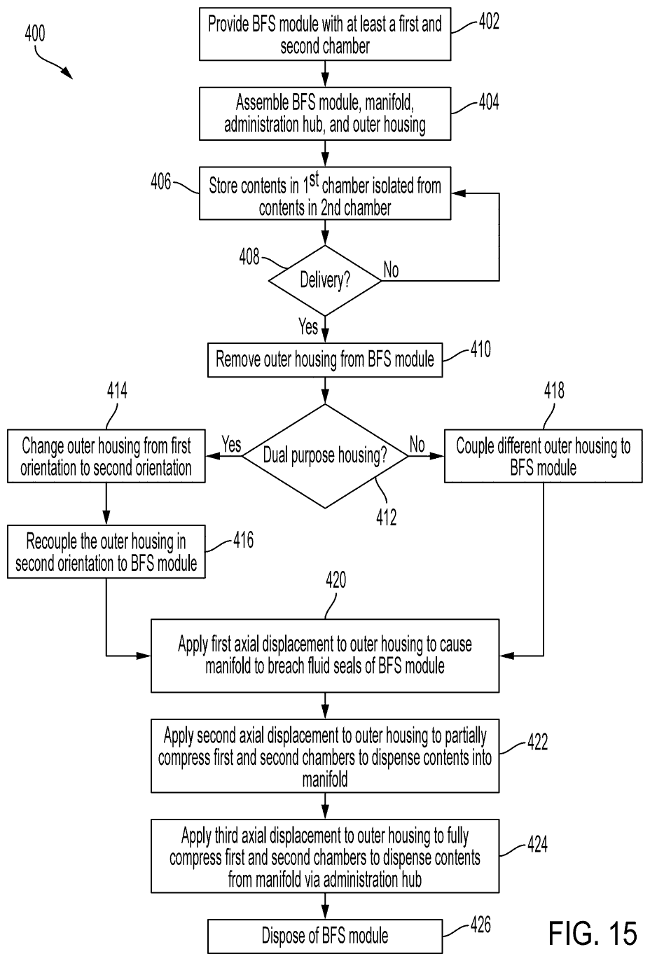

FIG. 15 illustrates an exemplary method 400 for administering a medical treatment using a pre-filled medical treatment system. The method 400 can initiate at process block 402, where a BFS module (e.g., a BFS vial) having at least two chambers separately storing respective fluid agents therein is provided. For example, the BFS module can be similar to any of the modules described above with the respect to FIGS. 1A-14B. In some embodiments, the provision of process block 402 can include manufacturing the BFS with the fluid agents sealed therein, for example, using a BFS manufacturing technique.

The method 400 can proceed to process block 404, where medical delivery assembly can be constructed, for example, by coupling together the BFS module, a manifold, and a casing (e.g. outer housing). For example, neck portions of the BFS module can be inserted into the manifold, such that mating features of the neck portions are received in corresponding first retaining features of the manifold, thereby retaining sealed ends of the neck portions aligned (e.g., spaced from or in non-breaching contact with) with piercing elements in the manifold. The casing in a first orientation (e.g., a protective configuration) can be advanced longitudinally over the part of the BFS module exposed from the manifold. In some embodiments, the casing in the first orientation can be releasably coupled to and retained on the manifold. In some embodiments, an administration assembly (e.g., administration hub 150 or 350, and administration member 156 or 356) can also to the medical delivery assembly, for example, by coupling to an outlet longitudinal end of the manifold.

The method 400 can proceed to process block 406, where the fluid agent contained in a first chamber of the BFS module is stored isolated from the fluid agent contained in a second chamber of the BFS module. In some embodiments, the storing of process block 406 can include providing appropriate environment conditions (e.g., a temperature at or below room temperature (e.g., 20-22° C.)) for the module to maintain viability of the fluid agents contained therein. For example, the BFS module can be maintained in an as-manufactured sealed configuration, with the contents of the first and second chambers separated from each other in the environment until combination of the separate liquid agents is desired (e.g., just prior to administration to a patient), as determined at decision block 408.

If administration of the fluid agents is desired at decision block 408, the method 400 can proceed to process block 410; otherwise, the method 400 returns to process block 406 to maintain the BFS module in a sealed state. At process block 410, the casing can be removed from the BFS module, for example, by displacing the casing longitudinally away from the manifold. The method 400 can then proceed from decision block 412 based on the construction and operation of the casing. If the casing is constructed as a dual-purpose housing (e.g., housing 360 that operates to provide protection or reservoir actuation depending on orientation), the method 400 can proceed to process block 414, where an orientation of the casing is changed (e.g., by flipping over or reversing) from a first orientation corresponding to a protective function to a second orientation corresponding to an actuation function. The re-oriented casing can have an internal actuation chamber extending from a longitudinal end thereof. The method 400 can then proceed to process block 416, where the re-oriented casing is re-coupled to the medical delivery assembly, for example, by advancing the internal actuation chamber onto the free end of the BFS module. Alternatively, the method 400 can proceed from decision block 412 to process block 418, where a second, different casing (or other actuation mechanism) can be used to actuate the BFS module. For example, the second casing (also referred to herein as administration casing or actuation casing) can have an internal actuation chamber extending from a longitudinal end thereof. The second casing can be coupled to the medical delivery assembly, for example, by advancing the internal actuation chamber onto the free end of the BFS module.

The method 400 can proceed to process block 420, where a longitudinal force applied to the casing can be transferred to the BFS module to cause the BFS module to experience a first axial displacement further into the manifold. For example, a user can longitudinally displace (e.g., by pressing) the casing (e.g., the reoriented casing or a second casing), to push the neck portions of the BFS module into contact with the piercing elements of the manifold, such that the sealed ports are pierced, broken, punctured, ruptured, or otherwise breached by the piercing elements, thereby providing fluid communication between the fluid-agent-containing chambers of the BFS module and the mixing chamber of the manifold. In some embodiments, the mating features of the neck portions are caused to move from the first retaining features of the manifold to adjacent second retaining features, thereby retaining the sealed ends of the neck portions with the piercing elements extending therethrough.

The method 400 can proceed to process block 422, where a longitudinal force applied to the casing that causes the casing to longitudinally advance (e.g., a second axial displacement) over part of the BFS module exposed from the manifold. For example, a user can longitudinally displace (e.g., by pressing) the casing (e.g., the reoriented casing or a second casing) toward the manifold such that the internal actuation chamber extends at least partially over and compresses the exposed part of the first and second reservoirs. The resulting pressure forces contents of the liquid-agent-containing chambers through the breached ports into the manifold. In some embodiments, the BFS module and the casing can be constructed such that the advancing of the casing over the BFS module in process block 422 can result an equal rate of dispensing (e.g., in terms of percentage of total fluid volume) from each reservoir. For example, the second axial displacement of the casing over the BFS module can result in 100% of the fluid agent in each reservoir being dispensed into the manifold at a same time. In some embodiments, the medical delivery assembly can be shaken after the contents are forced into the manifold, for example to ensure sufficient emptying of chambers of the BFS module and/or adequate mixing of the fluids in the mixing chamber of the manifold.

In some embodiments, after process block 420, the assembly may be ready for administration, and the administration member can be inserted into a patient before proceeding to process block 422. Alternatively, in some embodiments, the assembly is ready for administration after process block 422, and the administration member can be inserted into a patient before proceeding to process block 424. If the administration assembly includes a protective cap, it can be removed, and the administration member inserted into the patient. In some embodiments, the administration member can comprise a needle, and the inserting can comprise inserting an outlet end of the needle into the patient, for example, to effect a subcutaneous, intramuscular, intradermal, and intravenous injection of the combined liquid agents. Alternatively, in some embodiments, the administration member can comprise a nozzle, and the inserting can comprise inserting an outlet end of the nozzle into an orifice or cavity of the patient (e.g., oral, nasal, ear canal, etc.) or disposing adjacent to an exposed surface of the patient (e.g., for topical application, eyes, etc.)

The method 400 can proceed to process block 424, where the combined fluid agents contained in the mixing chamber of the manifold can be administered to a patient via the inserted administration member. For example, a user can further longitudinally displace (e.g., by pressing) the casing (e.g., the reoriented casing or a second casing) toward the manifold such that the internal actuation chamber further extends over and compresses the exposed part of the first and second reservoirs. The resulting pressure forces the contents in the mixing chamber out of the manifold through the administration member. In some embodiments, the dispensing of combined liquid agents of process block 424 is effective to a deliver a single dose of a therapeutic agent to the patient, for example, having a volume of 3 ml or less. After administration, the administration assembly can be removed from the patient.

The method 400 can proceed to process block 426, where some or all of the components of the pre-filled medical treatment assembly can be disposed. For example, the BFS module can be constructed for single use and may be incapable of (or at least undesirable for) reuse once the seals are breached and the liquid agents are dispensed therefrom. In some embodiments, at least the manifold of the mixing assembly can also be constructed for single use and may be discarded after the administration. In some embodiments, the casing can be retained for reuse, with or without cleaning and/or sterilization.

Although some of blocks 402-426 of method 400 have been described as being performed once, in some embodiments, multiple repetitions of a particular process block may be employed before proceeding to the next decision block or process block. In addition, although blocks 402-426 of method 400 have been separately illustrated and described, in some embodiments, process blocks may be combined and performed together (simultaneously or sequentially). Moreover, although FIG. 15 illustrates a particular order for blocks 402-426, embodiments of the disclosed subject matter are not limited thereto. Indeed, in certain embodiments, the blocks may occur in a different order than illustrated or simultaneously with other blocks. For example, in some embodiments, the assembly of process block 404 may occur after the storage of process block 406 and/or after the delivery decision of decision block 408, for example, where the medical delivery assembly is assembled in the field.

### VII. Additional Examples of the Disclosed Technology

In view of the above described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

Clause 1. A pre-filled multi-fluid medical delivery assembly, comprising:
a blow-fill-seal (BFS) module defining at least two collapsible fluid reservoirs, each collapsible fluid reservoir being in fluid communication with a respective neck comprising an external flange and each neck comprising a fluid sealed at a terminus thereof;
a mixing connector comprising a chamber into which each of the fluid reservoirs may be seated, a piercing element coupled to align with each sealed terminus of each neck of the BFS module in the case that the BFS module is inserted into the chamber, an interior volume defining a mixing chamber, and an outlet tube comprising threads and defining an outlet port;
a needle hub comprising threads that are cooperatively mated with the threads of the outlet tube of the mixing connector, the needle hub being coupled to a needle disposed through the needle hub;
a cap covering an administration end of the needle; and
a dual-purpose outer cover defining a first end comprising a first shaped chamber and a second end comprising a second shaped chamber, wherein the first shaped chamber is shaped to fit over the BFS module without compressing the collapsible fluid reservoirs and to removably couple to the mixing connector, and wherein the second shaped chamber is shaped to compress the collapsible fluid reservoirs in the case that the second end is forced onto the BFS module.

Clause 2. The assembly of any clause or example herein, in particular clause 1, wherein the mixing connector comprises, within the chamber, first and second flange seats arranged axially adjacent in the chamber and disposed to retain the external flanges of the necks of the BFS module in each of a first axial position and a second axial position.

Clause 3. The assembly of any clause or example herein, in particular, any one of clauses 1-2, wherein an insertion of the BFS module into the chamber in the second axial position causes the piercing elements to pierce the respective fluid seals of the BFS module, thereby releasing the fluids stored therein into the mixing chamber.

Clause 4. A pre-filled medical delivery assembly comprising:
(i) a blow-fill-seal (BFS) module having first and second longitudinal ends, the BFS module comprising:
   first and second reservoirs, the first reservoir having a first liquid agent therein, the second reservoir having a second liquid agent therein, the first and second reservoirs being spaced from each other along a lateral direction; and
   first and second sealed ports proximal to the first longitudinal end of the BFS module, the first sealed port being in fluid communication with the first reservoir, the second sealed port being in fluid communication with the second reservoir, the first and second sealed ports being spaced from each other along the lateral direction;
(ii) a manifold having third and fourth longitudinal ends, the manifold has an internal volume extending from the third longitudinal end to the fourth longitudinal end, a portion of the internal volume proximal to the third longitudinal end being constructed to act as a mixing chamber for the first and second liquid agents, the first longitudinal end of the BFS module being received through the fourth longitudinal end of the manifold with at least part of the first and second reservoirs exposed from the manifold, the manifold comprising first and second longitudinally-extending piercing elements disposed within the internal volume and respectively aligned with the first and second sealed ports; and
(iii) a removable casing disposed over the exposed part of the first and second reservoirs of the BFS module and releasably coupled to the manifold.

Clause 5. The assembly of any clause or example herein, in particular, clause 4, wherein the first and second reservoirs extend along a longitudinal direction.

Clause 6. The assembly of any clause or example herein, in particular, any one of clauses 4-5, wherein the removable casing is releasably coupled to the fourth longitudinal end of the manifold.

Clause 7. The assembly of any clause or example herein, in particular, any one of clauses 4-6, wherein the removable casing has (a) fifth and sixth longitudinal ends, and (b) an internal protection chamber extending from the fifth longitudinal end and accommodating therein the exposed part of the first and second reservoirs of the BFS module without compressing the first and second reservoirs.

Clause 8. The assembly of any clause or example herein, in particular, clause 7, wherein the internal protection chamber accommodates therein the exposed part of the first and second reservoirs of the BFS module without contacting the first and second reservoirs.

Clause 9. The assembly of any clause or example herein, in particular, any one of clauses 7-8, wherein a shape of the internal protection chamber in a cross-section parallel to the lateral direction is substantially the same as a shape of the first and second reservoirs in a cross-section parallel to the lateral direction.

Clause 10. The assembly of any clause or example herein, in particular, any one of clauses 7-9, wherein the removable casing further has an internal actuation chamber extending from the sixth longitudinal end, and/or the internal actuation chamber has an area in a cross-section parallel to the lateral direction that is less than that of the internal protection chamber.

Clause 11. The assembly of any clause or example herein, in particular, any one of clauses 4-10, further comprising an administration casing separate from the removable casing, the administration casing having (a) seventh and eighth longitudinal ends, and (b) an internal actuation chamber extending from the seventh longitudinal end and having an area in a cross-section parallel to the lateral direction that is less than that of the internal actuation chamber of the removable casing.

Clause 12. The assembly of any clause or example herein, in particular, any one of clauses 10-11, wherein the internal actuation chamber is constructed to compress the first and second reservoirs when displaced, along a longitudinal direction from the second longitudinal end of the BFS module, over the exposed part of the first and second reservoirs.

Clause 13. The assembly of any clause or example herein, in particular, any one of clauses 10-12, wherein the internal actuation chamber has a rectangular shape in the cross-section parallel to the lateral direction.

Clause 14. The assembly of any clause or example herein, in particular, clause 13, wherein a size of the rectangular shape is substantially constant along a longitudinal direction.

Clause 15. The assembly of any clause or example herein, in particular, any one of clauses 10-14, wherein the BFS module comprises a first flange laterally extending from the first reservoir and a second flange laterally extending from the second reservoir; the internal actuation chamber comprises first and second tracks on opposite lateral sides; and/or the first and second tracks are arranged to interface with the first and second flanges, respectively, when the internal actuation chamber is displaced over the exposed part of the first and second reservoirs.

Clause 16. The assembly of any clause or example herein, in particular, any one of clauses 4-15, wherein the BFS module has first and second necks, the first neck fluidically connecting the first reservoir to the first sealed port, the second neck fluidically connecting the second reservoir to the second sealed port; the manifold comprises first and second bores extending from the fourth longitudinal end, the first and second necks of the BFS module being received in the first and second bores, respectively; each of the first and second bores having a first mating feature; and/or each of the first and second necks has a laterally-protruding portion that is received in the first mating feature such that the first and second sealed ports are not breached by the first and second piercing elements.

Clause 17. The assembly of any clause or example herein, in particular, clause 16, the first mating feature comprises a scalloped portion of a sidewall of the respective bore, and each laterally-protruding portion has a toroidal shape in side view.

Clause 18. The assembly of any clause or example herein, in particular, any one of clauses 16-17, wherein each of the first and second bores further comprises a second mating feature, the second mating feature being disposed between the first mating feature and the mixing chamber; the laterally-protruding portion is constructed to laterally deform when longitudinally displaced from the first mating feature to the second mating feature; and/or with the laterally-protruding portion received in the second mating feature, the first and second piercing elements breach the first and second sealed ports, respectively.

Clause 19. The assembly of any clause or example herein, in particular, any one of clauses 4-18, wherein the manifold further comprises a plurality of guide portions at the fourth longitudinal end that form an interference fit with the removable casing.

Clause 20. The assembly of any clause or example herein, in particular, any one of clauses 4-19, wherein a volume of the first liquid agent is at least two times greater than a volume of the second liquid agent, and/or a volume of the first reservoir is at least two times greater than a volume of the second reservoir.

Clause 21. The assembly of any clause or example herein, in particular, any one of clauses 4-20, wherein the volume of the first liquid agent is about 2 ml, and the volume of the second liquid agent is about 0.7 ml.

Clause 22. The assembly of any clause or example herein, in particular, any one of clauses 4-21, wherein the first liquid agent, the second liquid agent, or both comprise a vaccine, a drug, a medicament, or a component of any of the foregoing.

Clause 23. The assembly of any clause or example herein, in particular, any one of clauses 4-22, wherein a seal of each of the first and second sealed ports comprises a foil, wax, paper, and/or a section of the BFS module.

Clause 24. The assembly of any clause or example herein, in particular, any one of clauses 4-23, further comprising (iv) an administration assembly comprising (a) a hub disposed proximal to the third longitudinal end of the manifold; and (b) at least one administration conduit having an outlet at an end thereof longitudinally spaced from the hub and being in fluid communication with the mixing chamber so as to receive a combination of the first and second liquid agents therefrom.

Clause 25. The assembly of any clause or example herein, in particular, clause 24, wherein at least part of the administration assembly is formed separate from and constructed to be coupled to the manifold, or at least part of the administration assembly is integrally formed with the manifold.

Clause 26. The assembly of any clause or example herein, in particular, any one of clauses 24-25, wherein the at least one administration conduit comprises a needle or cannula.

Clause 27. The assembly of any clause or example herein, in particular, any one of clauses 24-26, wherein the needle has a length in a range of 0.5 mm to 4 mm, inclusive, or in a range of 4 mm to 15 mm, inclusive, or in a range of 15 mm to 30 mm, inclusive.

Clause 28. The assembly of any clause or example herein, in particular, clause 24, wherein the outlet of the at least one administration conduit is formed as a nozzle configured to facilitate dispersion of the combination of the first and second liquid agents into a spray, or the outlet of the at least one administration conduit is formed as a nozzle configured to facilitate dispersion of the combination of the first and second liquid agents into one or more droplets.

Clause 29. The assembly of any clause or example herein, in particular, any one of clauses 4-28, wherein the removable casing, the manifold, or both are formed of a material having a hardness greater than that of the BFS module.

Clause 30. The assembly of any clause or example herein, in particular, any one of clauses 4-30, wherein the removable casing is formed of polypropylene, the manifold is formed of polycarbonate, and/or the BFS module is formed of low-density polyethylene (LDPE).

Clause 31. The assembly of any clause or example herein, in particular, any one of clauses 4-31, wherein each of the first and second longitudinally-extending piercing elements is substantially solid, such that the liquid agents from the first and second reservoirs flow around the respective piercing elements when the respective sealed ports are breached.

Clause 32. A kit comprising multiple components to be assembled to form the pre-filled medical delivery assembly of any clause or example herein, in particular, any one of clauses 1-31.

Clause 33. A method comprising:
coupling the manifold to the BFS module of a kit to form the pre-filled medical delivery assembly of any clause or example herein, in particular, any one of clause 1-32;
combining the first and second liquid agents; and
administering the combined liquid agents to a patient.

Clause 34. A method comprising:
(a) providing a blow-fill-seal (BFS) module having first and second longitudinal ends, the BFS module comprising:
   first and second reservoirs, the first reservoir having a first liquid agent therein, the second reservoir having a second liquid agent therein, the first and second reservoirs being spaced from each other along a lateral direction; and
   first and second sealed ports proximal to the first longitudinal end of the BFS module, the first sealed port being in fluid communication with the first reservoir, the second sealed port being in fluid communication with the second reservoir, the first and second sealed ports being spaced from each other along the lateral direction;
(b) inserting the first longitudinal end of the BFS module into a fourth longitudinal end of a manifold such that the first and second sealed ports are aligned with first and second longitudinally-extending piercing elements within the manifold and such that at least part of the first and second reservoirs are exposed from the manifold, the manifold defining an internal volume extending from a third longitudinal end thereof to the fourth longitudinal end, a portion of the internal volume proximal to the third longitudinal end defining a mixing chamber; and
(c) disposing a removable casing over the exposed part of the first and second reservoirs of the BFS module and releasably coupling the removable casing to the manifold, the removable casing having fifth and sixth longitudinal ends and an internal protection chamber that extends from the fifth longitudinal end, the internal protection chamber accommodating therein the exposed part of the first and second reservoirs of the BFS module without compressing the first and second reservoirs.

Clause 35. The method of any clause or example herein, in particular, clause 34, further comprising:
(d) removing the removable casing from the manifold and the BFS module;
(e) reorienting the removable casing such that the sixth longitudinal end of the casing faces the second longitudinal end of the BFS module, the casing further having an internal actuation chamber extending from the sixth longitudinal end;
(f) displacing the removable casing toward the manifold such that the internal actuation chamber contacts the exposed part of the first and second reservoirs so as to push the BFS module toward the third longitudinal end of the manifold and to breach the first and second sealed ports by the first and second piercing elements;
(g) further displacing the removable casing toward the manifold such that the internal actuation chamber extends at over and compresses the exposed part of the first and second reservoirs so as to dispense the first and second liquid agents from the first and second reservoirs into the mixing chamber via the breached ports; and
(h) further displacing the removable casing toward the manifold such that the internal actuation chamber extends over and compresses the exposed part of the first and second reservoirs so as to dispense the first and second liquid agents from the mixing chamber.

Clause 36. The method of any clause or example herein, in particular, clause 34, further comprising:
(d) removing the removable casing from the manifold and the BFS module;
(e) providing an administration casing with a seventh longitudinal end thereof facing the second longitudinal end of the BFS module, the administration casing having an internal actuation chamber extending from the seventh longitudinal end;
(f) displacing the administration casing toward the manifold such that the internal actuation chamber contacts the exposed part of the first and second reservoirs so as to push the BFS module toward the third longitudinal end of the manifold and to breach the first and second sealed ports by the first and second piercing elements;
(g) further displacing the administration casing toward the manifold such that the internal actuation chamber extends at over and compresses the exposed part of the first and second reservoirs so as to dispense the first and second liquid agents from the first and second reservoirs into the mixing chamber via the breached ports; and
(h) further displacing the administration casing toward the manifold such that the internal actuation chamber extends over and compresses the exposed part of the first and second reservoirs so as to dispense the first and second liquid agents from the mixing chamber.

Clause 37. A method comprising:
(a) providing a pre-filled medical delivery assembly comprising:
   (i) a blow-fill-seal (BFS) module having first and second longitudinal ends, the BFS module comprising:
      first and second reservoirs, the first reservoir having a first liquid agent therein, the second reservoir having a second liquid agent therein, the first and second reservoirs being spaced from each other along a lateral direction; and
      first and second sealed ports proximal to the first longitudinal end of the BFS module, the first sealed port being in fluid communication with the first reservoir, the second sealed port being in fluid communication with the second reservoir, the first and second sealed ports being spaced from each other along the lateral direction;
   (ii) a manifold having third and fourth longitudinal ends, the manifold has an internal volume extending from the third longitudinal end to the fourth longitudinal end, a portion of the internal volume proximal to the third longitudinal end being constructed to act as a mixing chamber for the first and second liquid agents, the first longitudinal end of the BFS module being received through the fourth longitudinal end of the manifold with at least part of the first and second reservoirs exposed from the manifold, the manifold comprising first and second longitudinally-extending piercing elements disposed within the internal volume and respectively aligned with the first and second sealed ports; and
   (iii) a casing disposed over the exposed part of the first and second reservoirs of the BFS module and releasably coupled to the manifold, the casing having fifth and sixth longitudinal ends, and an internal protection chamber extending from the fifth longitudinal end, the internal protection chamber accommodating therein the exposed part of the first and second reservoirs of the BFS module without compressing the first and second reservoirs.
(b) removing the casing from the manifold and the BFS module;
(c) reorienting the casing such that the sixth longitudinal end of the casing faces the second longitudinal end of the BFS module, the casing further having an internal actuation chamber extending from the sixth longitudinal end;
(d) displacing the casing toward the manifold such that the internal actuation chamber contacts the exposed part of the first and second reservoirs so as to push the BFS module toward the third longitudinal end of the manifold and to breach the first and second sealed ports by the first and second piercing elements;
(e) after (d), further displacing the casing toward the manifold such that the internal actuation chamber extends at least partially over and compresses the exposed part of the first and second reservoirs so as to dispense the first and second liquid agents from the first and second chambers into the mixing chamber via the breached ports; and
(f) after (e), further displacing the casing toward the manifold such that the internal actuation chamber extends over and compresses the exposed part of the first and second reservoirs so as to dispense the first and second liquid agents from the mixing chamber.

Clause 38. A method comprising:
(a) providing a pre-filled medical delivery assembly comprising:
   (i) a blow-fill-seal (BFS) module having first and second longitudinal ends, the BFS module comprising:
      first and second reservoirs, the first reservoir having a first liquid agent therein, the second reservoir having a second liquid agent therein, the first and second reservoirs being spaced from each other along a lateral direction; and
      first and second sealed ports proximal to the first longitudinal end of the BFS module, the first sealed port being in fluid communication with the first reservoir, the second sealed port being in fluid communication with the second reservoir, the first and second sealed ports being spaced from each other along the lateral direction;
   (ii) a manifold having third and fourth longitudinal ends, the manifold has an internal volume extending from the third longitudinal end to the fourth longitudinal end, a portion of the internal volume proximal to the third longitudinal end being constructed to act as a mixing chamber for the first and second liquid agents, the first longitudinal end of the BFS module being received through the fourth longitudinal end of the manifold with at least part of the first and second reservoirs exposed from the manifold, the manifold comprising first and second longitudinally-extending piercing elements disposed within the internal volume and respectively aligned with the first and second sealed ports; and
   (iii) a protective casing disposed over the exposed part of the first and second reservoirs of the BFS module and releasably coupled to the manifold, the protective casing having fifth and sixth longitudinal ends, and an internal protection chamber extending from the fifth longitudinal end, the internal protection chamber accommodating therein the exposed part of the first and second reservoirs of the BFS module without compressing the first and second reservoirs.
(b) removing the protective casing from the manifold and the BFS module;
(c) providing an administration casing with a seventh longitudinal end thereof facing the second longitudinal end of the BFS module, the administration casing having an internal actuation chamber extending from the seventh longitudinal end;
(d) displacing the administration casing toward the manifold such that the internal actuation chamber contacts the exposed part of the first and second reservoirs so as to push the BFS module toward the third longitudinal end of the manifold and to breach the first and second sealed ports by the first and second piercing elements;
(e) after (d), further displacing the administration casing toward the manifold such that the internal actuation chamber extends at least partially over and compresses the exposed part of the first and second reservoirs so as to dispense the first and second liquid agents from the first and second chambers into the mixing chamber via the breached ports; and
(f) after (e), further displacing the administration casing toward the manifold such that the internal actuation chamber extends over and compresses the exposed part of the first and second reservoirs so as to dispense the first and second liquid agents from the mixing chamber.

Clause 39. The method of any clause or example herein, in particular, any one of clauses 34-38, further comprising coupling a hub of an administration assembly to the third longitudinal end of the manifold, the administration assembly further comprising at least one administration conduit having an outlet at an end thereof longitudinally spaced from the hub and being in fluid communication with the mixing chamber so as to receive a combination of the first and second liquid agents therefrom.

Clause 40. The method of any clause or example herein, in particular, clause 39, wherein the at least one administration conduit comprises a needle or cannula, and the method further comprises inserting an outlet end of the needle or cannula into a patient.

Clause 41. The method of any clause or example herein, in particular, clause 40, wherein the needle has a length in a range of 0.5 mm to 4 mm, inclusive, or in a range of 4 mm to 15 mm, inclusive, or in a range of 15 mm to 30 mm, inclusive.

Clause 42. The method of any clause or example herein, in particular, any one of clauses 39-41, wherein the administering is via subcutaneous, intramuscular, intradermal, and intravenous injection of the combined liquid agents.

Clause 43. The method of any clause or example herein, in particular, clause 39, wherein the outlet of the at least one administration conduit is formed as a nozzle configured to facilitate dispersion of the combination of the first and second liquid agents into a spray, or the outlet of the at least one administration conduit is formed as a nozzle configured to facilitate dispersion of the combination of the first and second liquid agents into one or more droplets, and the method further comprises inserting an outlet end of the nozzle into an orifice of a patient.

Clause 44. The method of any clause or example herein, in particular, any one of clauses 33-43, wherein the first liquid agent, the second liquid agent, or both comprise a vaccine, a drug, a medicament, or a component of any of the foregoing.

Clause 45. The method of any clause or example herein, in particular, any one of clauses 33-44, wherein a seal of each of the first and second sealed ports comprises a foil, wax, paper, a section of the BFS module, or any combination of the foregoing.

Clause 46. The method of any clause or example herein, in particular, any one of clauses 33-45, wherein a volume of the first liquid agent is at least two times greater than a volume of the second liquid agent; a volume of the first reservoir is at least two times greater than a volume of the second reservoir; a volume of the second liquid agent is less than a volume of the first liquid agent; and/or a volume of the first liquid agent is 2 ml or less.

Clause 47. The method of any clause or example herein, in particular, any one of clauses 33-46, further comprising, prior to the coupling, filling the first and second liquid agents in the first and second chambers, respectively, using a blow-fill-seal technique.

Clause 48. The method of any clause or example herein, in particular, any one of Clauses 33-47, wherein the liquid agents provide a single dose of a therapeutic agent to the patient.

### VIII. Rules of Interpretation

Any of the features illustrated or described with respect to FIGS. 1A-15 and Clauses 1-48 can be combined with any other features illustrated or described with respect to FIGS. 1A-15 and Clauses 1-48 to provide systems, assemblies, kits, devices, methods, and embodiments not otherwise illustrated or specifically described herein. For example, the nozzle for the administration assembly can be applied to any other manifold or administration assembly, described herein or otherwise, for example, the manifold 330 of FIGS. 3A-14B, or the administration hub 350 of FIGS. 5A-14B. Other combinations and variations are also possible according to one or more contemplated embodiments. All features described herein are independent of one another and, except where structurally impossible, can be used in combination with any other feature described herein.

Any or all of the components disclosed herein can be formed of one or more plastics. In some embodiments, some components (e.g., the BFS vials) can be formed of a relatively soft polymer (e.g., having a Shore/Durometer “D” hardness of between 60 and 70), such as polyethylene (e.g., low density polyethylene (LDPE)), polypropylene, or any other polymer adaptable for use in a BFS manufacturing process. In some embodiments, some components (e.g., the connection assemblies, the administration assemblies, and/or needle caps or covers) can be formed, at least in part, of a relatively hard polymer (e.g., having a hardness greater than 80 on the Rockwell “R” scale), such as, but not limited to, polypropylene, polycarbonate, polybenzimidazole, acrylonitrile butadiene styrene (ABS), polystyrene, polyvinyl chloride, or the like. Other materials are also possible according to one or more contemplated embodiments.

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments.

Neither the Title (set forth at the beginning of the first page of this patent application) nor the Abstract (set forth at the end of this patent application) is to be taken as limiting in any way as the scope of the disclosed invention(s).

While the term “modules” is utilized herein for convenience and ease of illustration, objects represented and/or described as “modules” may comprise various forms, configurations, and/or quantities of components. A BFS module may comprise one or more BFS products that are formed and/or manufactured together or separately, for example, and/or may comprise one or more BFS chambers, bottles, containers, and/or other fluid-retaining objects. The term “module” does not convey any designation of shape or size. In some embodiments, a BFS module may comprise one or more vials. According to some embodiments a BFS module and/or a BFS vial may comprise one or more fluid chambers. In some embodiments, a plurality of BFS modules, components, vials, and/or chambers may be manufactured simultaneously from a single BFS mold. Each respective module and/or chamber may be formed, for example, by different portions of a single BFS mold (e.g., two cooperative halves thereof). In some embodiments, BFS modules, components, vials, and/or chambers may be joined and/or coupled during manufacturing (e.g., via unformed and/or fused connecting parison) and/or after manufacturing/filling.

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this patent application and the title of this patent application are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining and the like

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pre-filled medical delivery assembly comprising:
(i) a blow-fill-seal (BFS) module having first and second longitudinal ends, the BFS module comprising:
first and second reservoirs, the first reservoir having a first liquid agent therein, the second reservoir having a second liquid agent therein, the first and second reservoirs being spaced from each other along a lateral direction; and
first and second sealed ports proximal to the first longitudinal end of the BFS module, the first sealed port being in fluid communication with the first reservoir, the second sealed port being in fluid communication with the second reservoir, the first and second sealed ports being spaced from each other along the lateral direction;
(ii) a manifold having third and fourth longitudinal ends, the manifold has an internal volume extending from the third longitudinal end to the fourth longitudinal end, a portion of the internal volume proximal to the third longitudinal end being constructed to act as a mixing chamber for the first and second liquid agents, the first longitudinal end of the BFS module being received through the fourth longitudinal end of the manifold with at least part of the first and second reservoirs exposed from the manifold, the manifold comprising first and second longitudinally-extending piercing elements disposed within the internal volume and respectively aligned with the first and second sealed ports; and
(iii) a removable casing disposed over the exposed part of the first and second reservoirs of the BFS module and releasably coupled to the manifold.

2. The pre-filled medical delivery assembly of claim 1, wherein the first and second reservoirs extend along a longitudinal direction.

3. The pre-filled medical delivery assembly of claim 1, wherein the removable casing is releasably coupled to the fourth longitudinal end of the manifold.

4. The pre-filled medical delivery assembly of claim 1, wherein the removable casing has:
fifth and sixth longitudinal ends; and
an internal protection chamber extending from the fifth longitudinal end and accommodating therein the exposed part of the first and second reservoirs of the BFS module without compressing the first and second reservoirs.

5. The pre-filled medical delivery assembly of claim 4, wherein the internal protection chamber accommodates therein the exposed part of the first and second reservoirs of the BFS module without contacting the first and second reservoirs.

6. The pre-filled medical delivery assembly of claim 4, wherein a shape of the internal protection chamber in a cross-section parallel to the lateral direction is substantially the same as a shape of the first and second reservoirs in a cross-section parallel to the lateral direction.

7. The pre-filled medical delivery assembly of claim 4, wherein the removable casing further has an internal actuation chamber extending from the sixth longitudinal end, the internal actuation chamber having an area in a cross-section parallel to the lateral direction that is less than that of the internal protection chamber.

8. The pre-filled medical delivery assembly of claim 1, further comprising an administration casing separate from the removable casing, the administration casing having:
seventh and eighth longitudinal ends; and
an internal actuation chamber extending from the seventh longitudinal end and having an area in a cross-section parallel to the lateral direction that is less than that of the internal actuation chamber of the removable casing.

9. The pre-filled medical delivery assembly of claim 1, wherein:
the BFS module has first and second necks, the first neck fluidically connecting the first reservoir to the first sealed port, the second neck fluidically connecting the second reservoir to the second sealed port;
the manifold comprises first and second bores extending from the fourth longitudinal end, the first and second necks of the BFS module being received in the first and second bores, respectively;
each of the first and second bores having a first mating feature; and
each of the first and second necks has a laterally-protruding portion that is received in the first mating feature such that the first and second sealed ports are not breached by the first and second piercing elements.

10. The pre-filled medical delivery assembly of claim 9, wherein the first mating feature comprises a scalloped portion of a sidewall of the respective bore, and each laterally-protruding portion has a toroidal shape in a side view.

11. The pre-filled medical delivery assembly of claim 9, wherein:
each of the first and second bores further comprises a second mating feature, the second mating feature being disposed between the first mating feature and the mixing chamber;
the laterally-protruding portion is constructed to laterally deform when longitudinally displaced from the first mating feature to the second mating feature; and
with the laterally-protruding portion received in the second mating feature, the first and second piercing elements breach the first and second sealed ports, respectively.

12. The pre-filled medical delivery assembly of claim 1, wherein the manifold further comprises a plurality of guide portions at the fourth longitudinal end that form an interference fit with the removable casing.

13. The pre-filled medical delivery assembly of claim 1, wherein:
a volume of the first liquid agent is at least two times greater than a volume of the second liquid agent;
a volume of the first reservoir is at least two times greater than a volume of the second reservoir; or
both of the above.

14. The pre-filled medical delivery assembly of claim 13, wherein the volume of the first liquid agent is about 2 ml, and the volume of the second liquid agent is about 0.7 ml.

15. The pre-filled medical delivery assembly of claim 1, wherein the first liquid agent, the second liquid agent, or both comprise a vaccine, a drug, a medicament, or a component of any of the foregoing.

16. The pre-filled medical delivery assembly of claim 1, wherein a seal of each of the first and second sealed ports comprises a foil, wax, paper, a section of the BFS module, or any combination of the foregoing.

17. The pre-filled medical delivery assembly of claim 1, further comprising:
(iv) an administration assembly comprising:
a hub disposed proximal to the third longitudinal end of the manifold; and
at least one administration conduit having an outlet at an end thereof longitudinally spaced from the hub and being in fluid communication with the mixing chamber so as to receive a combination of the first and second liquid agents therefrom.

18. The pre-filled medical delivery assembly of claim 17, wherein at least part of the administration assembly is formed separate from and constructed to be coupled to the manifold, or at least part of the administration assembly is integrally formed with the manifold.

19. The pre-filled medical delivery assembly of claim 17, wherein the at least one administration conduit comprises a needle or cannula.

20. The pre-filled medical delivery assembly of claim 17, wherein the needle has a length in a range of 0.5 mm to 4 mm, inclusive, or in a range of 4 mm to 15 mm, inclusive, or in a range of 15 mm to 30 mm, inclusive.
